(19) United States
(12) Patent Application Publication
(10) Pub. No.: US 2024/0310375 A1
(43) Pub. Date: Sep. 19, 2024

(54) BIOPARTICLE ANALYSIS METHOD AND REAGENT KIT FOR BIOPARTICLE ANALYSIS

(71) Applicant: Sony Group Corporation, Tokyo (JP)

(72) Inventor: Masahiro Matsumoto, Kanagawa (JP)

(73) Assignee: Sony Group Corporation, Tokyo (JP)

(21) Appl. No.: 18/279,482

(22) PCT Filed: Feb. 4, 2022
(86) PCT No.: PCT/JP2022/004477

§ 371 Date: Aug. 30, 2023

(30) Foreign Application Priority Data
Mar. 10, 2021 (JP) 2021-038639

(51) Int. Cl. G01N 33/569

(52) U.S. Cl. CPC G01N 33/56972; G01N 2469/10

(57) ABSTRACT

An object of the present invention is to provide a technique for analyzing a bioparticle that is included in a bioparticle population and that is in a state in which an influence of the interparticle interaction is reflected.

The present disclosure provides a bioparticle analysis method including: a preparation step of preparing a bioparticle population including a bioparticle to which a first capturing substance configured to capture a secreted substance is bound; a first capturing step of binding a secreted substance generated by placing the bioparticle population under a predetermined condition, to the first capturing substance; and a second capturing step of binding the secreted substance bound to the first capturing substance, to a second capturing substance configured to capture the secreted substance. The present disclosure also provides a reagent kit for bioparticle analysis used in the analysis method, and a bioparticle analysis system used for executing the analysis method.

### TECHNICAL FIELD

The present disclosure relates to a bioparticle analysis method and a reagent kit for bioparticle analysis. More specifically, the present disclosure relates to a bioparticle analysis method for performing single cell analysis of each bioparticle included in a bioparticle population, and a reagent kit for bioparticle analysis used in the analysis method.

### BACKGROUND ART

In order to analyze the reactivity of cells, it has been proposed to measure secreted molecules. For example, Patent Document 1 below discloses a method for identifying a cell population including effector cells having an extracellular effect. Patent Document 1 describes, as steps included in the method, a step of retaining a cell population including one or more effector cells in a microreactor containing a readout particle population including one or more readout particles, and a step of incubating the cell population and the one or more readout particles in the microreactor (claim 1). Patent Document 1 describes that the extracellular effect is a direct or indirect effect on a readout particle that is extracellular of an effector cell. Patent Document 1 also describes, as a more specific example, that the extracellular effect is the binding of a target biomolecule secreted by an effector cell to a readout particle, or is a response such as apoptosis of a readout cell or an accessory cell (paragraph 0183).

In addition, Patent Document 2 below discloses a method for analyzing a secreted protein. Patent Document 2 describes that the method includes: encapsulating a cell in a microdrop containing a predetermined component; binding a molecule secreted from the cell to a capturing molecule, thereby retaining the secreted molecule in the microdrop; and detecting the secreted molecule (claim 1).

### CITATION LIST

### Patent Document

Patent Document 1: Japanese Translation of PCT Application No. 2016-515823
Patent Document 2: Japanese Translation of PCT Application No. 2004-528574

### SUMMARY OF THE INVENTION

### Problems to be Solved by the Invention

An object of the present disclosure is to provide a technique for analyzing a bioparticle in a state of being included in a bioparticle population, particularly a single cell analysis technique for a cell in a state of being included in a cell population.

### Solutions to Problems

The present disclosure provides a bioparticle analysis method including:
a preparation step of preparing a bioparticle population including a bioparticle to which a first capturing substance configured to capture a secreted substance is bound;
a first capturing step of binding a secreted substance generated by placing the bioparticle population under a predetermined condition, to the first capturing substance; and
a second capturing step of binding the secreted substance bound to the first capturing substance, to a second capturing substance configured to capture the secreted substance.

The first capturing step may include a treatment step of placing the bioparticle population under a predetermined condition, and
the treatment step may be performed while a population state of the bioparticle population is maintained.

The first capturing step and the second capturing step may be performed while a state in which the first capturing substance is bound to the bioparticle is maintained.

A particle identifier configured to identify the bioparticle may be bound to the bioparticle included in the bioparticle population prepared in the preparation step.

A capturing substance identifier configured to identify the second capturing substance may be bound to the second capturing substance.

The first capturing substance may include a secreted substance binding part and a bioparticle binding part.

The secreted substance binding part may be configured to bind to one or two or more of the secreted substances.

The bioparticle binding part may contain an antigen binding substance that binds to an antigen on a surface of the bioparticle, or a molecule binding substance that binds to a molecule forming a surface membrane of the bioparticle.

The secreted substance binding part may be bound to the bioparticle binding part via a crosslinking part.

The first capturing substance may contain an antibody that binds to surfaces of two or more cells of the same or different types.

The bioparticle analysis method according to the present disclosure may further include an isolation step of isolating the bioparticle included in the bioparticle population into a single particle after the second capturing step.

The bioparticle analysis method according to the present disclosure may further include a disruption step of disrupting the bioparticle after the isolation step.

The disruption step may be performed under an environment in which a component contained in one bioparticle is not mixed with a component contained in another bioparticle.

The bioparticle analysis method according to the present disclosure may further include an analysis step of analyzing each of the bioparticles after the disruption step.

Furthermore, the present disclosure also provides
a reagent kit for bioparticle analysis, the reagent kit including:
a first secreted substance capturing substance including: a first bioparticle binding part configured to bind to a bioparticle; and a first secreted substance binding part configured to bind to a secreted substance generated by placing a bioparticle population including the bioparticle under a predetermined condition; and
a second secreted substance capturing substance including: a second secreted substance binding part configured to bind to the secreted substance; and a capturing substance identifier configured to identify a second capturing substance.

The first secreted substance capturing substance may further include a crosslinking part that crosslinks the bioparticle binding part and the secreted substance binding part.

The first bioparticle binding part may contain an antigen binding substance that binds to an antigen on a surface of the bioparticle, or a molecule binding substance that binds to a molecule forming a surface membrane of the bioparticle.

The antigen binding substance may contain a substance selected from the group consisting of an antibody, an antibody fragment, an aptamer, and a molecularly imprinted polymer.

The molecule binding substance may include an oleyl group or a cholesteryl group.

The reagent kit may further include a base material having a surface on which a particle capturing substance including: a second bioparticle binding part configured to bind to the bioparticle; and a particle identifier configured to identify the bioparticle is immobilized.

### BRIEF DESCRIPTION OF DRAWINGS

FIG. 1A is an example of a flowchart of a bioparticle analysis method of the present disclosure.

FIG. 1B is an example of a flowchart of a production step.

FIG. 2A is a schematic view for illustrating the production step.

FIG. 2B is a schematic view for illustrating a first capturing step.

FIG. 2C is a schematic view for illustrating a second capturing step.

FIG. 2D is a schematic view for illustrating an isolation step, a disruption step, and an analysis step.

FIG. 3A is a schematic view for illustrating a particle capturing substance.

FIG. 3B is a view illustrating an example of a molecule binding substance.

FIG. 4 is a schematic view for illustrating a first capturing substance.

FIG. 5 is a schematic view for illustrating a bioparticle to which a first capturing substance and a particle capturing substance are bound.

FIG. 6 is a schematic view for illustrating a second capturing substance.

FIG. 7A is a view illustrating an example of a microchip used for forming emulsion particles.

FIG. 7B is a schematic view for illustrating isolation of bioparticles in emulsion particles.

FIG. 8 is a schematic enlarged view of a particle sorting part.

FIG. 9 is a schematic enlarged view of the particle sorting part.

FIG. 10 is an example of a flowchart of a method for forming an emulsion.

FIG. 11A is a schematic enlarged view of a connection channel part.

FIG. 11B is a schematic enlarged view of the connection channel part.

FIG. 12A is a schematic enlarged view of the connection channel part.

FIG. 12B is a schematic enlarged view of the connection channel part.

FIG. 13 is a schematic view for illustrating a state in which a container is connected to the microchip.

FIG. 14 is a schematic view of another example of the microchip.

FIG. 15 is a schematic view of an example of a well used for executing a particle isolation step.

FIG. 16 is a schematic view for illustrating that a bioparticle-containing droplet is generated by a nozzle provided in a microfluidic chip.

FIG. 17 is a schematic view illustrating an example of a state in which a first capturing substance is bound to a bioparticle.

FIG. 18 is a schematic view illustrating an example of a state in which a first capturing substance is bound to a bioparticle.

FIG. 19 is a schematic view illustrating an example of a state in which a first capturing substance is bound to a bioparticle.

FIG. 20 is a schematic view illustrating a state in which two cells are captured by one first capturing substance.

FIG. 21 is a schematic view illustrating an example of a first capturing substance containing antibodies that bind to two or more bioparticles.

FIG. 22 is a schematic view for illustrating an example of crosslinking of two or more bioparticles.

FIG. 23 is a schematic view for illustrating an example of crosslinking of two or more bioparticles.

FIG. 24 is a schematic view illustrating a state in which surface molecule binding substances are bound to a bioparticle.

FIG. 25 is a schematic view for illustrating a surface molecule binding substance to which an identification substance is bound.

### MODE FOR CARRYING OUT THE INVENTION

Hereinafter, preferred embodiments for carrying out the present disclosure will be described. Note that embodiments described below illustrate representative embodiments of the present disclosure, and the scope of the present disclosure is not limited only to these embodiments. Note that the present disclosure will be described in the following order.
1. First embodiment (bioparticle analysis method)
(1) Description of problems
(2) Description of first embodiment
(3) Examples of first embodiment
(3-1) Preparation step
(3-1-1) Surface preparation step
(3-1-2) Surface capturing step
(3-1-3) Capturing substance binding step
(Modified Example 1: embodiment in which secreted substance binding part contains a plurality of secreted substance binding substances)
(Modified Example 2: embodiment in which bioparticle binding part is multispecific antibody)
(Modified Example 3: first capturing substance containing antibody that binds to two or more bioparticles)
(Modified Example 4: crosslinking of two or more bioparticles)
(3-1-4) Cleavage step
(3-1-4-1) Detection step
(3-1-4-2) Linker cleavage step
(3-2) First capturing step
(3-3) Second capturing step
(Modified Example 5: use of substance that binds to surface molecule of bioparticle)
(3-4) Isolation step
(3-4-1) Discrimination step
(3-4-2) Particle isolation step
(3-4-2-1) Case of space in emulsion particle
(3-4-2-2) Case of space in well
(3-5) Disruption step
(3-6) Analysis step
2. Second embodiment (reagent kit for bioparticle analysis)
3. Third embodiment (bioparticle analysis system)

### 1. First Embodiment (Bioparticle Analysis Method)

### (1) Description of Problems

As described above, some techniques for analyzing the reactivity of cells by measuring secreted molecules have been proposed. However, these techniques do not take into account effects due to interactions between cells in a cell population (for example, immune cell population) that includes a plurality of types of cells.

In addition, the correlation between the gene expression and the secreted molecular weight may be low. Therefore, only measuring intracellular molecules may be insufficient for analysis of cells. For more detailed analysis of cells, it is considered desirable to simultaneously measure intracellular molecules and extracellular secreted molecules, and further simultaneously measure cell surface molecules in addition to these molecules.

With respect to a cell population including a plurality of types of cells, for example, an immune cell population, it is difficult to simultaneously specify cell types of cells included in the cell population, analyze intracellular molecules included in the cells, and analyze extracellular molecules (particularly, secreted molecules) related to the cells.

In order to perform these specifying and/or analysis, use of a fluorescent dye as a label is conceivable. However, due to the overlapping of the fluorescence spectra, the number of types of molecules that can be identified in the case of using the fluorescent dye is about several tens at most. The cell type can be specified by flow cytometry, but it is difficult to obtain other information (for example, information regarding the intracellular molecules and/or the extracellular secreted molecules) using only the fluorescent dye.

In addition, in order to analyze extracellular molecules secreted from cells, beads configured to capture the molecules may be used. In this case, it is conceivable to isolate the cells and the beads in a microspace, and then capture the molecules. However, in a case where a plurality of types of cells is contained in a sample, it is difficult to specify the cells that have secreted the molecules, and the molecules secreted from certain cells.

In light of the above, a main object of the present disclosure is to provide a technique for analyzing a bioparticle in a state of being included in a bioparticle population. Another object of the present disclosure is to provide a technique for analyzing one or more substances (particularly, secreted substances) present outside the bioparticle and/or one or more substances present inside the bioparticle. The analysis may be performed, for example, on each bioparticle included in the bioparticle population.

### (2) Description of First Embodiment

A method according to the present disclosure includes: a preparation step of preparing a bioparticle population including a bioparticle to which a first capturing substance configured to capture a secreted substance is bound; a first capturing step of binding a secreted substance generated by placing the bioparticle population under a predetermined condition, to the first capturing substance; and a second capturing step of binding the secreted substance bound to the first capturing substance, to a second capturing substance configured to capture the secreted substance. As a result, it is possible to form a state in which the secreted substance generated in a case where the bioparticle population is placed under a predetermined condition is captured by the first capturing substance and the second capturing substance, and these three substances (the secreted substance, the first capturing substance, and the second capturing substance) are bound to the bioparticle. It is therefore possible to analyze a bioparticle in a state in which the interparticle interaction in the bioparticle population is reflected. That is, in the method according to the present disclosure, the first capturing step and the second capturing step may be performed while a state in which the first capturing substance is bound to the bioparticle is maintained.

The present disclosure is suitable for analyzing cells included in a cell population having diversity, such as an immune cell population. For example, according to the present disclosure, it is possible to obtain information regarding cells (the type or state of cells, for example, the degree of differentiation, and the like) reflecting the influence of intercellular interactions in a cell population, and information regarding extracellular molecules (particularly, secreted substances). According to the present disclosure, in addition to these pieces of information, information regarding intracellular molecules can also be obtained. For example, according to the present disclosure, it is possible to directly or indirectly observe which cell among cells included in a cell population having a certain cell configuration is reacting, by analysis of secreted substances (for example, specifying the type of the secreted substance or measurement of the amount of the secreted substance). As a result, it is possible to elucidate the function of a diverse cell population.

In the method according to the present disclosure, the secreted substance may be captured by the first capturing substance bound to the surface of the bioparticle. Furthermore, in the method according to the present disclosure, the bioparticle is not necessarily in an isolated state in order to cause a reaction that generates the secreted substance, and the reaction may be performed in an environment in which a plurality of types of bioparticles exists.

In addition, the secreted substance captured on the surface of the bioparticle reacts with the second capturing substance (for example, a secreted substance binding antibody to which a capturing substance identifier such as an oligo barcode is bound). The second capturing substance can be analyzed or measured with, for example, a particle identifier (including an oligo barcode or the like) bound to the surface of the bioparticle. Therefore, the secreted substance can also be analyzed or measured by associating the secreted substance with the second capturing substance in advance. Moreover, in addition to the analysis or measurement of the secreted substance, the analysis of the surface antigen of the bioparticle and/or the analysis of the gene expression in the bioparticle can be executed simultaneously. In addition, it is possible to confirm from which bioparticle the secreted substance secreted by placing the bioparticle population under the condition that promotes secretion of the secreted substance is derived, for example, with the particle identifier bound to the surface of the bioparticle.

In a preferred embodiment, the first capturing step includes a treatment step of placing the bioparticle population under a predetermined condition, and the treatment step is performed while a population state of the bioparticle population is maintained. As a result, it is possible to perform a reaction where the secreted substance is generated while the intercellular interaction in the cell population is maintained. Then, after the reaction, the cell type, cell state, intracellular gene expression, extracellular secreted molecules, and the like can be analyzed with single cell resolution.

The method according to the present disclosure may further include an isolation step of isolating the bioparticle included in the bioparticle population into a single particle after the second capturing step. The method according to the present disclosure may further include a disruption step of disrupting the bioparticle after the isolation step. The disruption step may be executed while the isolated state is maintained. That is, the disruption step may be performed under an environment in which a component contained in one bioparticle is not mixed with a component contained in another bioparticle. The method according to the present disclosure may further include an analysis step of analyzing each of the bioparticles after the disruption step.

Through these steps, the reactivity of cells included in a cell population including a plurality of types of cells can be analyzed with single cell resolution. The functionality of each cell in a certain cell population can be clarified by this analysis. Also in this analysis, it is possible to specify cells or cell populations optimal for treatment in an in vitro assay. Therefore, the present disclosure contributes to improvement in the response rate of a cell population (for example, a cell therapeutic agent) used for treatment of diseases such as cancer.

### (3) Examples of First Embodiment

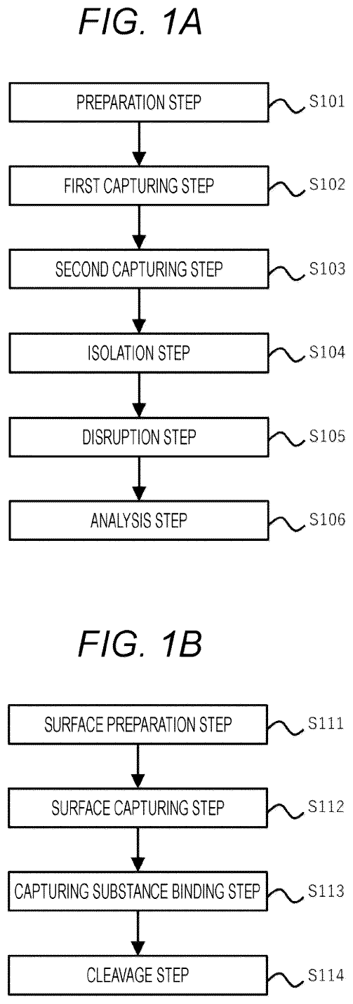

A bioparticle analysis method of the present disclosure will be described below with reference to FIG. 1A. FIG. 1A is an example of a flowchart of the bioparticle analysis method.

For example, as illustrated in FIG. 1A, the bioparticle analysis method of the present disclosure includes a preparation step S101, a first capturing step S102, a second capturing step S103, an isolation step S104, a disruption step S105, and an analysis step S106. Each step is described below.

### (3-1) Preparation Step

In the preparation step S101, a bioparticle population including a bioparticle to which a first capturing substance for capturing a secreted substance is bound is prepared. The bioparticle population may be, for example, a cell population. The cell population is, for example, an immune cell population or a blood cell population.

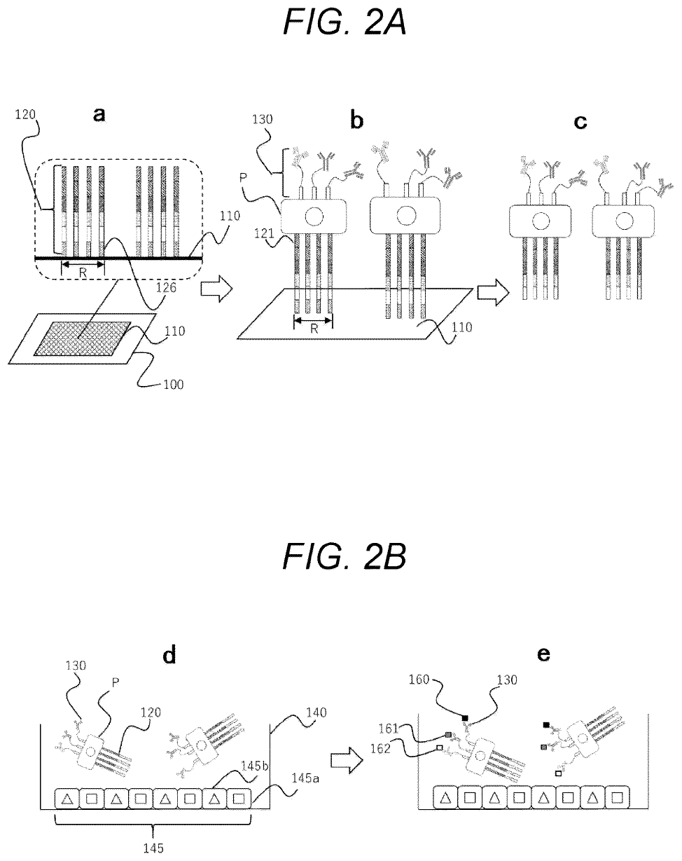

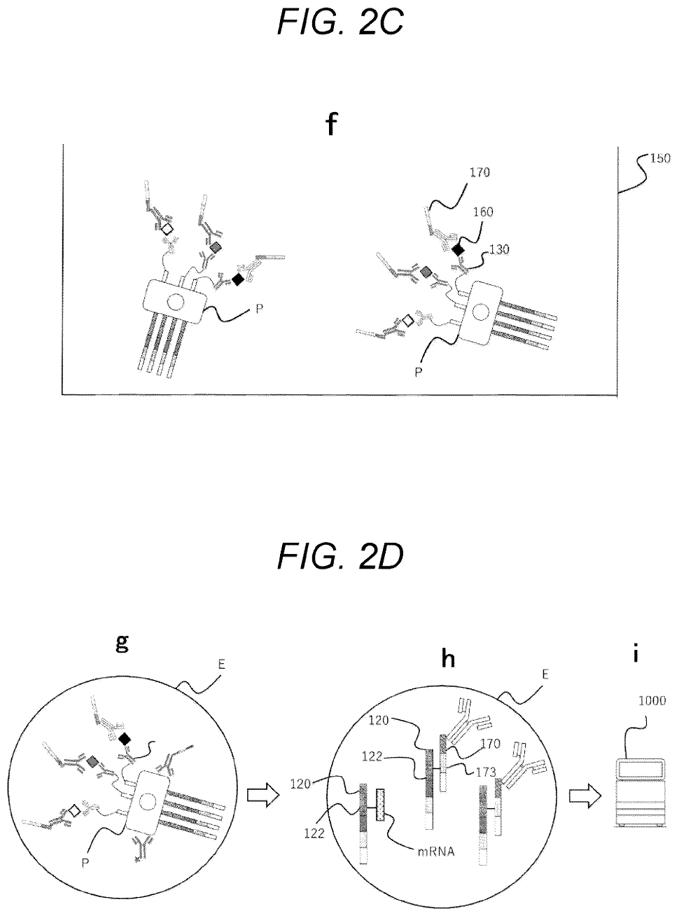

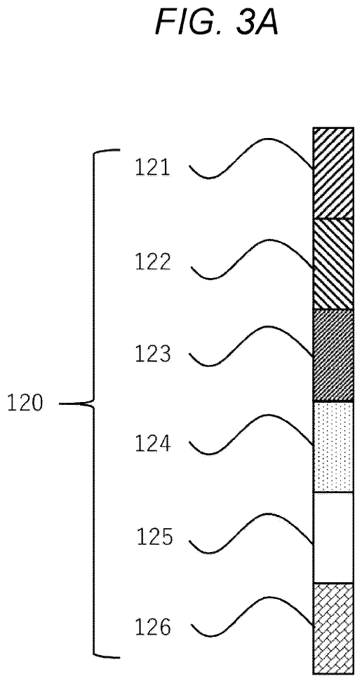

The preparation step includes a production step of the bioparticle population. An example of the production step will be described with reference to FIGS. 1B and 2A. FIG. 1B is an example of a flowchart of the production step. FIG. 2A is a schematic view for illustrating the production step.

As illustrated in FIG. 1B, the production step may include a surface preparation step S111, a surface capturing step S112, a capturing substance binding step S113, and a cleavage step S114. These steps will be described below.

### (3-1-1) Surface Preparation Step

In the surface preparation step S111, a surface on which a particle capturing substance is immobilized is prepared. For example, as illustrated in a of FIG. 2A, a plurality of particle capturing substances 120 is immobilized on a surface 110 of a substrate 100.

The particle capturing substance 120 is immobilized to the surface 110 via a linker 126 included as a part of the substance.

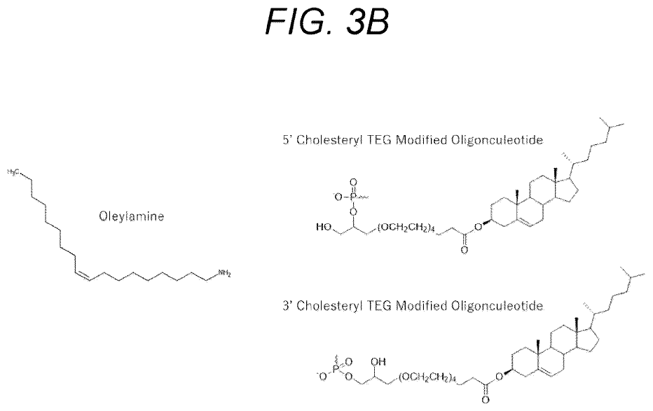

In addition to the linker 126, as illustrated in FIG. 3A, the particle capturing substance 120 further includes a particle capturing part 121, a substance recovery part 122 (for example, poly T), a unique molecular identifier (UMI) part 123, a particle identifier 124 (for example, a cell barcode), and a recovered substance amplification part 125 (for example, a primer for nucleic acid amplification and/or a promoter for nucleic acid transcription). These will be described below.

The particle capturing part 121 is configured to capture a bioparticle, and is particularly configured to capture a cell. The particle capturing part 121 may be a bioparticle binding substance. The bioparticle binding substance may be an antigen binding substance that binds to an antigen on the surface of a bioparticle P, or a molecule binding substance that binds to a molecule forming a surface membrane of the bioparticle P.

The antigen binding substance may contain a substance selected from the group consisting of an antibody, an antibody fragment, an aptamer, and a molecularly imprinted polymer. The antibody or the antibody fragment may be, for example, an antibody or an antibody fragment that binds to a component (particularly, a surface antigen) present on the surface of a bioparticle such as a cell. The aptamer may be a nucleic acid aptamer or a peptide aptamer. The aptamer and the molecularly imprinted polymer may also bind to, for example, a component (particularly, a surface antigen) present on the surface of a bioparticle such as a cell.

The molecule binding substance is, for example, a compound having an oleyl group or a cholesteryl group. These groups can non-specifically bind to a molecule forming a surface membrane of the bioparticle P (for example, a cell). The oleyl group and the cholesteryl group may bind to, for example, a bioparticle including a lipid bilayer membrane, such as a cell. Examples of the compound having an oleyl group include oleylamine illustrated on the left of FIG. 3B. Examples of the compound containing a cholesteryl group include cholesterol-TEG (a triethylene glycol spacer having 15 atoms) illustrated on the right of FIG. 3B. The upper right of FIG. 3B illustrates a state in which cholesterol-TEG is bound to the 5′ end of the oligonucleotide. The lower right of FIG. 3B illustrates a state in which cholesterol-TEG is bound to the 3′ end of the oligonucleotide. By modifying the oligonucleotide with a molecule binding substance including an oleyl group or a cholesteryl group, the oligonucleotide can capture a bioparticle.

The substance recovery part 122 is configured to capture a conjugate of the first capturing substance, the secreted substance, and the second capturing substance formed in the item (3-3) second capturing step described later and/or a molecule contained in the bioparticle.

The substance recovery part 122 may contain, for example, a nucleic acid or a protein.

The nucleic acid may be configured to comprehensively capture the conjugate, and mRNA contained in the bioparticle (particularly, a cell), and may be, for example, a poly T sequence. The poly T sequence can bind to a poly A sequence contained in the second capturing substance constituting the conjugate. In addition, the poly T sequence can also bind to a poly A sequence contained in mRNA in the bioparticle.

Alternatively, the nucleic acid may have a sequence complementary to a target sequence contained in the conjugate or a target sequence of the nucleic acid in the bioparticle. The nucleic acid can bind to these target sequences by having the complementary sequence.

In a case where the substance recovery part is a protein, the protein may be, for example, an antibody. The substance recovery part may be an aptamer or a molecular imprinted polymer.

The substance recovery part 122 may contain two or more types of constituent elements for capturing the conjugate or the molecule contained in the bioparticle. The substance recovery part 122 may contain both a protein and a nucleic acid, and may contain, for example, both an antibody and a poly T sequence. Thereby, both a protein and mRNA can be detected simultaneously.

The unique molecular identifier (UMI) part 123 may contain a nucleic acid, particularly may contain DNA or RNA, and more particularly contains DNA. The UMI part 123 may have a sequence of, for example, 5 bases to 30 bases, particularly 6 bases to 20 bases, and more particularly 7 bases to 15 bases.

The UMI part 123 may have different sequences among the particle capturing substances immobilized to the surface 110. For example, in a case where the UMI part has a nucleic acid sequence of 10 bases, the type of the UMI sequence is 4¹⁰, that is, 1 million or more.

The UMI part 123 may be used for quantifying molecules contained in the bioparticle. For example, in a case where a molecule to be quantified is mRNA, a UMI sequence may be added to cDNA obtained by reverse transcription of mRNA as a target substance, for example, in the analysis step described later. Multiple CDNAs obtained by amplifying cDNA reverse transcribed from one mRNA molecule have the same UMI sequences, but multiple CDNAs obtained by amplifying cDNA transcribed from another mRNA molecule having the same sequence as the sequence of the one mRNA have different UMI sequences. Therefore, the number of copies of mRNA can be determined by counting the number of types of UMI sequences having the same cDNA sequence. Therefore, the analysis step described later may include, for example, determining the number of copies of mRNA, or may include counting the number of types of UMI sequences having the same DNA sequence.

For example, the UMI part 123 may have sequences different among a plurality of particle capturing substances that contain the same particle identifier and that are immobilized in one region R (for example, a spot or bead described later) illustrated in a and b of FIG. 2A. That is, a plurality of target capturing molecules immobilized to the region R (for example, a spot or bead described later) may have the same particle identifier, but may have different UMI parts (in particular, UMI parts having base sequences different from each other).

The particle identifier 124 is used for identifying or specifying a bioparticle to which the particle identifier has been bound (more specifically, a bioparticle to which the particle capturing substance including the particle identifier has been bound). The particle identifier 124 contains, for example, a nucleic acid having a barcode sequence. The nucleic acid may be particularly DNA or RNA, and more particularly DNA. The barcode sequence may be used, for example, for specifying a captured bioparticle (in particular, a cell), and in particular, may be used as an identifier for making a bioparticle isolated in a certain microspace distinguishable from a bioparticle isolated in another microspace. The barcode sequence may also be used as an identifier for making a particle capturing substance including a certain barcode sequence distinguishable from a particle capturing substance including another barcode sequence. The barcode sequence may be associated with a bioparticle to which the particle capturing substance including the barcode sequence is bound. Furthermore, the barcode sequence may be associated with information regarding a position on the surface 110 on which the particle capturing substance including the barcode sequence is immobilized. Moreover, the barcode sequence may be associated with a microspace in which the bioparticle to which the particle capturing substance including the barcode sequence is bound is isolated, and further, may be associated with information regarding the position of the microspace.

The information regarding the position is, for example, information regarding XY coordinates, but is not limited thereto. An ID number may be assigned to the barcode sequence associated with the position information. The ID number may be used in steps subsequent to the cleavage step. The ID number may correspond to the barcode sequence on a one-to-one basis, and may be used as data corresponding to the barcode sequence in steps subsequent to the cleavage step.

As described above, a particle identifier for identifying the bioparticle may be bound to the bioparticle included in the bioparticle population prepared in the preparation step S101.

A plurality of particle capturing substances immobilized in a certain region of the surface 110 may have the same particle identifier (in particular, the same barcode sequence). As a result, the certain region and the particle identifiers are associated with each other. By setting the size of the certain region to be smaller than the size of the bioparticle, the particle capturing substance including the particle identifier can be associated with the position where one bioparticle exists. For example, as illustrated in a and b of FIG. 2A, the region R on which a plurality of particle capturing substances 120 including the same particle identifier is immobilized may be smaller than the size of the bioparticle P.

As described above, the surface 110 used in the bioparticle analysis method of the present disclosure may have a plurality of regions on which a plurality of particle capturing substances having the same particle identifier is immobilized. Then, the particle identifier may be different for each region. The size (for example, the maximum dimension of the region, the diameter, the long diameter, the length of the long side, or the like of the region) of each region may be preferably smaller than the size of the bioparticle. The size of each region may be, for example, 50 μm or less, preferably 10 μm or less, and more preferably 5 μm or less.

The plurality of regions may be arranged at intervals such that, for example, a bioparticle captured by a particle capturing substance immobilized in one region is not captured by a particle capturing substance immobilized in another region. The interval may be, for example, a distance equal to or longer than the size of the bioparticle, and may be preferably a distance larger than the size of the bioparticle.

The number of the plurality of regions is preferably larger than the number of bioparticles applied to the surface 110 in the capturing step. Such a configuration prevents two or more bioparticles from being captured in one region.

In one embodiment of the present disclosure, a particle capturing substance including a known particle identifier (particularly, a barcode sequence whose sequence is known) may be immobilized in a predetermined region. For example, the surface 110 has a plurality of regions, and a plurality of particle capturing substances immobilized to each of the plurality of regions may include the same particle identifier. The plurality of regions may be set to be smaller than the size of the bioparticle to be captured. With the surface 110 having such a configuration, each of the plurality of regions can be associated with the particle identifier included in the plurality of target capturing molecules immobilized to each region.

A region where the particle capturing substances including the same particle identifier are immobilized as described above is also referred to as a spot in the present specification. That is, the size of the spot may be, for example, 50 μm or less, preferably 10 μm or less, and more preferably 5 μm or less.

The surface 110 configured as described above can associate a particle identifier included in a certain particle capturing substance with a position where the certain particle capturing substance exists when the particle capturing substance is immobilized on the surface 110. For the immobilization, for example, biotin is bound to the linker 1 of the particle capturing substance, streptavidin is bound to the surface 101 on which the particle capturing substance is to be immobilized, and then, the biotin and the streptavidin are bound, whereby the particle capturing substance is immobilized on the surface 110.

In another embodiment of the present disclosure, the particle capturing substance including the particle identifier may be randomly arranged on the surface 110.

In this case, after the particle capturing substance including the particle identifier is immobilized to the surface 110, the particle identifier included in the immobilized particle capturing substance is specified (in particular, the barcode sequence is read). Thereby, a particle identifier included in a certain particle capturing substance is associated with a position where the certain particle capturing substance exists. The reading can be performed by, for example, a technique such as sequencing by synthesis, sequencing by ligation, or sequencing by hybridization.

In addition, the particle identifier included in a certain particle capturing substance and the position where the certain particle capturing substance exists are not necessarily associated with each other. In this case, for example, in the isolation step described later, the bioparticle and the particle capturing substance are separated in a microspace. Thereby, the bioparticle and the particle capturing substance (particularly, the barcode sequence contained in the particle capturing substance) are associated on a one-to-one basis.

In this embodiment, for example, beads (for example, gel beads) to which a plurality of particle capturing substances including the same particle identifier is bound may be used. The beads (for example, gel beads) may be immobilized to, for example, the surface 110. The size of the beads (for example, gel beads) may be, for example, 50 μm or less, preferably 10 μm or less, and more preferably 5 μm or less. In order to bind the particle capturing substance to the beads (for example, gel beads), a combination of biotin and streptavidin may be used, for example. For example, biotin is bound to the linker 126 of the particle capturing substance, streptavidin is bound to the beads, and then the biotin and the streptavidin are bound. Thereby, the particle capturing substance is immobilized on the beads.

The surface 110 may be provided with a plurality of recesses. One spot or one bead in the embodiment may be arranged in each of the plurality of recesses. The spots or the beads can be more easily arranged on the surface 110 by the plurality of recesses. The size of the recess is preferably a size in which one bead is placed, for example. The shape of the recess may be, but is not limited to, a circle, an ellipse, a hexagon, or a quadrangle.

In addition, in the surface 110, the state of the surface portion on which the spot or the bead is arranged may be different from another surface portion. For example, the surface portion on which the spot or the bead is arranged may be hydrophilic, and the other surface portion may be hydrophobic, or the other surface portion may be hydrophobic and have a protrusion. Examples of a technique for imparting hydrophilicity to the surface include reactive ion etching in the presence of oxygen, irradiation with deep ultraviolet light in the presence of ozone, and the like. In these techniques, a mask having a penetrated portion corresponding to a portion to which hydrophilicity is to be imparted may be used. In addition, examples of a technique for imparting hydrophobicity to the surface include a silicone spray (spray-on-silicone), and for example, Techspray 2101-12S or the like may be used. Even in the case of imparting hydrophobicity, for example, a mask having a penetrated portion corresponding to a portion to which hydrophobicity is to be imparted may be used.

The particle capturing substance can also be synthesized on a substrate by, for example, a DNA microarray production technique or the like. The particle capturing substance can be synthesized at a specific position by, for example, a technique used for photolithography, such as a digital micromirror device (DMD), a liquid crystal shutter, or a spatial light modulator. Techniques for the synthesis are described, for example, in Basic Concepts of Microarrays and Potential Applications in Clinical Microbiology, CLINICAL MICROBIOLOGY REVIEWS, October 2009, page. 611 to 633. Incidentally, in the case of synthesizing the particle capturing substance on the substrate by the synthesis, information regarding the position where the particle capturing substance is synthesized is acquired at the time of synthesizing the particle capturing substance, and the particle identifier and the position information are associated with each other. At that time, an ID number may be assigned to each particle identifier.

In one embodiment of the present disclosure, all of the particle capturing substances immobilized on the surface may include a common oligo sequence. By using a fluorescently labeled nucleic acid having a sequence complementary to the oligo sequence, it is possible to confirm a position where the particle capturing substance is immobilized (particularly, the position of the spot or the position of the bead), and in particular, to confirm the position in the dark field. Furthermore, in a case where there is no recess or protrusion described above on the surface, it may be difficult to grasp the position where the particle capturing substance is immobilized. In this case, the fluorescent label makes it easy to grasp the position where the particle capturing substance is immobilized.

The recovered substance amplification part 125 may contain, for example, a nucleic acid having a primer sequence used for amplification of a nucleic acid and/or a promoter sequence used for transcription of a nucleic acid in the analysis step described later. The nucleic acid may be DNA or RNA, and is particularly DNA. The recovered substance amplification part 125 may have both a primer sequence and a promoter sequence. The primer sequence may be, for example, a PCR handle. The promoter sequence may be, for example, a T7 promoter sequence. In the present specification, the recovered substance amplification part 125 is also referred to as a first recovered substance amplification part in order to be distinguished from a second recovered substance amplification part 172 described later.

The linker 126 may be a linker cleavable by stimulation, and is, for example, a linker cleavable by light stimulation or chemical stimulation. Light stimulation is particularly suitable for selectively stimulating specific positions in the cleavage step described later.

The linker 126 may contain, for example, any one selected from an arylcarbonylmethyl group, a nitroaryl group, a coumarin-4-ylmethyl group, an arylmethyl group, a metal-containing group, and other groups, as a linker cleavable by light stimulation. As these groups, those described in, for example, Photoremovable Protecting Groups in Chemistry and Biology: Reaction Mechanisms and Efficacy, Chem. Rev. 2013, 113, page 119 to 191 may be used.

The arylcarbonylmethyl group may be, for example, a phenacyl group, an o-alkylphenacyl group, or a p-hydroxyphenacyl group. The nitroaryl group may be, for example, an o-nitrobenzyl group, an o-nitro-2-phenethyloxycarbonyl group, or o-nitroanilide. The arylmethyl group may be, for example, one into which a hydroxy group is introduced, or one into which no hydroxy group is introduced.

In a case where the linker 126 is a linker cleavable by light stimulation, the linker may be cleaved by light having a wavelength of preferably 360 nm or more. The linker may be a linker that is preferably cleaved at an energy of 0.5 μJ/μm² or less. (Light-sheet fluorescence microscopy for quantitative biology, Nat Methods. 2015 January; 12 (1): 23-6. doi: 10.1038/nmeth.3219.). By adopting a linker cleaved by light having the above wavelength or the above energy, it is possible to reduce cell damage (particularly, cleavage of DNA or RNA and the like) that may occur when a light stimulus is applied.

Particularly preferably, the linker may be a linker cleaved by light in a short wavelength range, specifically light in a wavelength range of 360 nm to 410 nm, or may be a linker cleaved by light in the near infrared region or the infrared region, specifically light in a wavelength range of 800 nm or more. In a case where the linker is a linker that is efficiently cleaved by light having a wavelength in the visible light region, it may be difficult to handle the analysis surface. Therefore, the linker is preferably a linker cleaved by the light in the short wavelength range, or the light in the near infrared region or the infrared region.

The linker 126 may contain, for example, a disulfide bond or a restriction endonuclease recognition sequence, as a linker cleavable by chemical stimulation. For cleavage of the disulfide bond, for example, a reducing agent such as tris(2-carboxyethyl) phosphine (TCEP), dithiothreitol (DTT), or 2-mercaptoethanol is used. For example, in a case where TCEP is used, the reaction is performed using 50 mM of TCEP for about 15 minutes, for example. For dissociation of the restriction endonuclease recognition sequence, an appropriate restriction endonuclease (http://catalog.takara-bio.co.jp/product/basic_info.php?unitid=U100003632) is used according to each sequence. 1 U of the restriction endonuclease activity is the amount of enzyme that completely degrades 1 μg of λDNA per hour at 37° C. in principle in 50 μl of each enzyme reaction solution. The amount of enzyme is adjusted according to the amount of the restriction endonuclease recognition sequence.

In order to increase the efficiency in the cleavage step described later, the particle capturing substance 120 may include a plurality of cleavable linkers. Preferably, the plurality of linkers may be connected in series. For example, in a case where the cleavage probability of one linker is 0.8, the cleavage probability is improved to 0.992 (=1−0.2³) by connecting three linkers in series.

### (3-1-2) Surface Capturing Step

In the surface capturing step S112, for example, as illustrated in b of FIG. 2A, the bioparticle P is captured by the particle capturing substance 120. In particular, the bioparticle is captured by the particle capturing part 121 of the particle capturing substance 120. In the surface capturing step S112, the bioparticle and the particle capturing part 121 may bind to each other in a specific or non-specific manner.

For example, in a case where the bioparticle is a cell, the cell may be captured by the particle capturing substance 120 through binding of a surface antigen of the cell and an antibody, an aptamer, or a molecularly imprinted polymer contained in the particle capturing part 121. The antibody, the aptamer, and the molecularly imprinted polymer may be specific or non-specific to the surface antigen. Also in this case, the cell may be captured by the particle capturing substance 120 through binding of the lipid bilayer membrane of the cell and an oleyl group or a cholesteryl group included in the particle capturing part 121.

The surface capturing step S112 may include an application step of applying the bioparticle to the surface 110. The application form may be performed, for example, by bringing a sample containing a bioparticle population (for example, a bioparticle-containing liquid) into contact with the surface 110. For example, a sample containing a bioparticle population may be dropped onto the surface 110.

In the surface capturing step S112, preferably, the plurality of particle capturing substances bound to one bioparticle may have the same particle identifier. Thereby, one particle identifier (in particular, a barcode sequence) can be associated with one bioparticle. In addition, preferably, the UMI parts included in the plurality of particle capturing substances may have base sequences different from each other. Thereby, the number of copies of mRNA can be determined, for example.

The surface capturing step S112 may include an incubation step for binding the bioparticle and the particle capturing substance. Incubation conditions such as incubation time and temperature may be determined according to the types of the bioparticle and the particle capturing substance to be used.

After the surface capturing step S112 is executed, a removal step of removing unnecessary substances such as bioparticles that have not bound to the particle capturing substance 120 may be performed. The removal step may include washing the surface 110 with a liquid, for example, a buffer.

### (3-1-3) Capturing Substance Binding Step

As illustrated in b of FIG. 2A, a first capturing substance 130 for capturing a secreted substance is bound to each bioparticle. The number of types of the first capturing substance 130 to be bound to one bioparticle may be one or more. Furthermore, the number of the first capturing substances 130 to be bound to one bioparticle may be one or more, but is preferably plural.

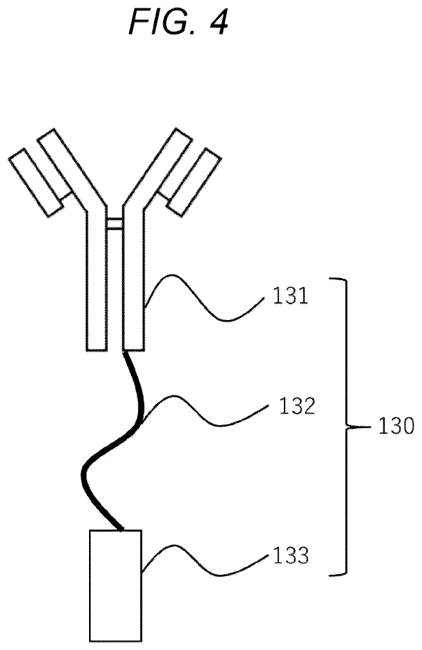

The first capturing substance 130 will be described with reference to FIG. 4. FIG. 4 is a schematic view for illustrating an example of the structure of the first capturing substance 130. As illustrated in the drawing, the first capturing substance 130 includes a secreted substance binding part 131 and a bioparticle binding part 133. The first capturing substance 130 further includes a crosslinking part 132. The secreted substance binding part 131 is bound to the bioparticle binding part 133 via the crosslinking part 132.

The secreted substance binding part 131 may be configured to bind to one or two or more secreted substances. The secreted substance binding part 131 may be appropriately designed or produced by those skilled in the art according to the secreted substance to be bound. For example, the secreted substance binding part 131 may be, for example, a substance selected from the group consisting of an antibody, an antibody fragment, an aptamer, and a molecularly imprinted polymer, and is particularly an antibody or an antibody fragment. Note that, in FIG. 4, an antibody is illustrated as the secreted substance binding part 131. The binding of the secreted substance binding part 131 may be specific or non-specific, and is particularly specific. The number of types of the secreted substance binding part 131 bound to one bioparticle P may be one or more.

The secreted substance to which the secreted substance binding part 131 binds is a secreted substance generated by placing a bioparticle population including the bioparticle P under a predetermined condition. The secreted substance may be a substance secreted from the bioparticle P, a substance secreted from another bioparticle included in the bioparticle population, or a secreted substance derived from an environment constituting the predetermined condition. The environment constituting the predetermined condition may be appropriately selected by the user who executes the bioparticle analysis method of the present disclosure, and may be an environment containing a material for which the reactivity of the bioparticle population is analyzed. The environment may be, for example, an environment in which the bioparticle population is incubated, and is, for example, an environment in a medium or a buffer solution. The material for which the reactivity of the bioparticle population is analyzed may be selected according to the reactivity to be analyzed, and may be a biomaterial or a non-biomaterial. The biomaterial may be, for example, a diseased tissue, a diseased cell, a microorganism (bacteria, fungus, or virus), or a heterologous tissue. The non-biomaterial may be, for example, a drug or a toxic substance. The diseased tissue may be, for example, a tumor tissue, and in particular, may be a cancer tissue or a sarcoma tissue. The diseased cell may be, for example, a tumor cell, and in particular, may be a cancer cell, a sarcoma cell, or a malignant lymphoma cell.

For example, in a case where the bioparticle population is an immune cell population and the reactivity of the immune cell population with a diseased tissue or a diseased cell is analyzed, the environment constituting the predetermined condition may be a liquid material (particularly, a medium or a buffer solution) containing the diseased tissue or the diseased cell.

The crosslinking part 132 is a substance that crosslinks the secreted substance binding part 131 and the bioparticle binding part 133. Note that the bioparticle binding part 133 may be directly bound to the secreted substance binding part 131, and in this case, the first capturing substance 130 need not include the crosslinking part.

The crosslinking part 132 may be, for example, the compounds described in International Publication No. 2017/177065, or a stereoisomer, a salt, or a tautomer thereof. The compound will be described below.

The crosslinking part 132 may be a compound having the following structure (I):

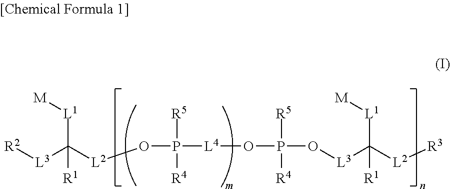

or a stereoisomer, a salt or a tautomer thereof. The secreted substance binding part 131 may be bound to any one of R² and R³ in the structure (I), and the bioparticle binding part 133 may be bound to the other.

In the structure (I):
M is independently at each occurrence a moiety having two or more carbon-carbon double bonds and at least one degree of conjugation;
L¹ is independently at each occurrence i) an optional alkylene, alkenylene, alkynylene, heteroalkylene, heteroalkenylene, heteroalkynylene, or heteroatom linker; or ii) a linker having a functional group capable of being formed by a reaction of two complementary reactive groups;
L² and L³ are independently at each occurrence an optional alkylene, alkenylene, alkynylene, heteroalkylene, heteroalkenylene, heteroalkynylene, or heteroatom linker;
L⁴ is independently at each occurrence a heteroalkylene, heteroalkenylene, or heteroalkynylene linker having a length greater than 3 atoms, where the heteroatom in the heteroalkylene, heteroalkenylene, and heteroalkynylene linker is selected from O, N, and S;
R¹ is independently at each occurrence H, alkyl, or alkoxy;
R² and R³ are each independently H, OH, SH, alkyl, alkoxy, alkyl ether, heteroalkyl, —OP(═Rₐ)(R_{b})R_{c}, Q, or a protected form thereof, or L′;
R⁴ is independently at each occurrence OH, SH, O⁻, S⁻, OR_{d}, SR_{d}, or Q;
R⁵ is independently at each occurrence oxo, thioxo, or absent;
Rₐ is O or S;
R_{b} is OH, SH, O⁻, S⁻, OR_{d} or SR_{d};
R_{c} is OH, SH, O⁻, S⁻, OR_{d}, OL′, SR_{d}, alkyl, alkoxy, heteroalkyl, heteroalkoxy, alkyl ether, alkoxyalkyl ether, phosphate, thiophosphate, phosphoalkyl, thiophosphoalkyl, phosphoalkyl ether, or thiophosphoalkyl ether;
R_{d} is a counter ion;
Q is independently at each occurrence a moiety having a reactive group capable of forming a covalent bond with an analyte molecule, a targeting moiety, a solid support or a complementary reactive group Q′, or a protected form thereof;
L′ is independently at each occurrence a linker having a covalent bond to Q, a linker having a covalent bond to a targeting moiety, a linker having a covalent bond to an analyte molecule, a linker having a covalent bond to a solid support, a linker having a covalent bond to a solid support residue, a linker having a covalent bond to a nucleoside, or a linker having a covalent bond to another compound of the structure (I);
m is independently at each occurrence an integer of 0 or greater, provided that at least one occurrence of m is an integer of 1 or greater; and
n is an integer of 1 or more.

Furthermore, the secreted substance binding part 131 may be bound to R⁴ in the structure (I). For example, the bioparticle binding part 133 may be bound to any one of R² and R³, and the secreted substance binding part 131 may be bound to the other one of R² and R³ and each of one or two or more selected from R⁴s. An example of the structure to which a plurality of secreted substance binding parts 131 is bound is also described for reference in Modified Example 1 described later.

In order to bind a plurality of the same or different secreted substance binding parts to R⁴, a moiety in which a secreted substance binding part is attached to an R⁴ moiety of the structure (I) is prepared (hereinafter referred to as R⁴⁻¹), and similarly, R⁴⁻², R⁴⁻³, . . . , and R⁴⁻ⁱ (here, i may be, for example, an integer of 2 to 500, particularly 2 to 300, more particularly 2 to 100, 2 to 50, 2 to 20, or 2 to 10, or even more particularly an integer of 2 to 4) are prepared. Then, R⁴⁻¹ to R⁴⁻ⁱ may be sequentially incorporated into the structure (I), for example, as in DNA synthesis.

Among R⁴⁻¹ to R⁴⁻ⁱ, R⁴ (also referred to as R⁴⁻⁰) to which no secreted substance binding part is bound may be introduced as a spacer. For example, one or more P atoms to which R⁴⁻⁰ having no secreted substance binding part is bound may exist between one P atom to which R⁴ having a secreted substance binding part is bound and another P atom to which R⁴ having a secreted substance binding part is bound.

In addition, R⁴ may include a spacer molecule such as PEG, that is, the atom P and the secreted substance binding part may be bound via the spacer molecule.

Regarding the compound having the structure (I), L⁴ may be independently at each occurrence an alkylene oxide linker.

Regarding the compound having the structure (I), L⁴ is polyethylene oxide, and the compound has the following structure (IA):

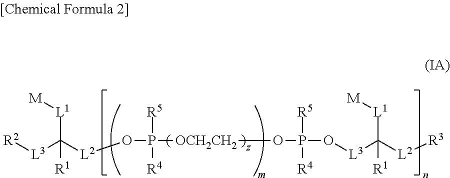

where z may be an integer of 2 to 100, for example, an integer of 3 to 6.

Regarding the compound having the structure (I), L¹ may have one of the following structures:

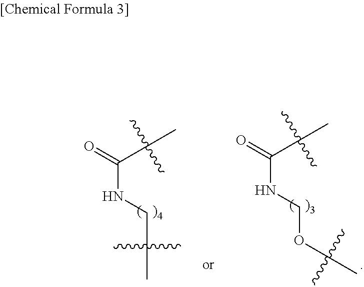

The compound may have the following structure (IB):

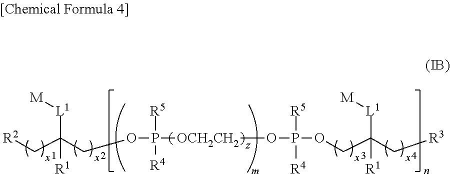

where
x¹, x², x³, and x⁴ are independently at each occurrence integers of 0 to 6, and
z may be an integer of 2 to 100.
x¹ and x³ may each be 0 at each occurrence, and x² and x⁴ may each be 1 at each occurrence.
x¹, x², x³, and x⁴ may each be 1 at each occurrence.

Regarding the compound having the structure (I), R⁴ may be independently at each occurrence OH, O⁻ or OR_{d}, and R⁵ may be at each occurrence oxo.

Regarding the compound having the structure (I), R¹ may be at each occurrence H.

Regarding the compound having the structure (I), R² and R³ may each independently be —OP(═Rₐ)(R_{b})R_{c}.

R_{c} may be OL′.

L′ may be a heteroalkylene linker to Q, a targeting moiety, an analyte molecule, a solid support, a solid support residue, a nucleoside, or another compound of the structure (I).

L′ may include an alkylene oxide or a phosphodiester moiety, or a combination thereof.

L′ has the following structure:

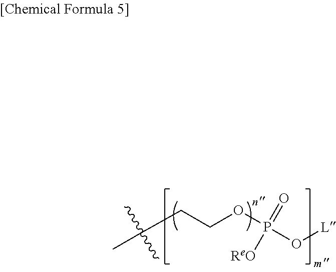

where
m″ and n″ are independently an integer of 1 to 10; and
R^{e} is H, an electron pair, or a counter ion; and
L″ may be R^{e} or a direct bond, or may be a linkage to Q, a targeting moiety, an analyte molecule, a solid support, a solid support residue, a nucleoside, or another compound of the structure (I).

The targeting moiety may be an antibody or a cell surface receptor antagonist.

Regarding the compound having the structure (I), R² or R³ may have one of the following structures:

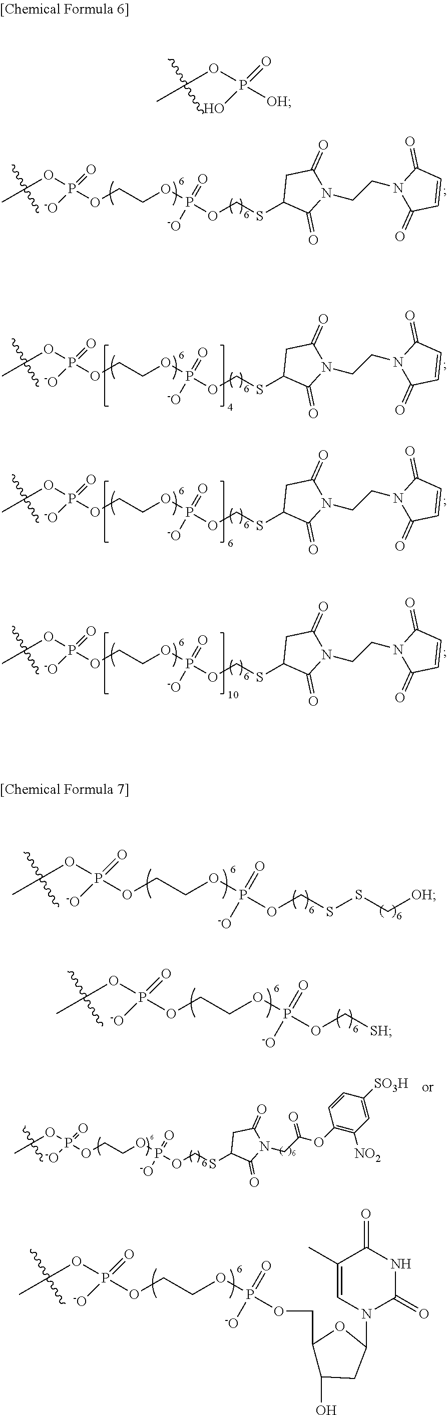

Regarding the compound having the structure (I), Q may include sulfhydryl, disulfide, activated ester, isothiocyanate, azide, alkyne, alkene, diene, dienophile, acid halide, sulfonyl halide, phosphine, α-haloamide, biotin, amino, or maleimide functional group.

Q may include a maleimide functional group.

Regarding the compound having the structure (I), Q may include a moiety selected from the structures of Table 1 (Tables 1-1 to 1-3) below.

TABLE 1-1
Table 1. Representative Q moiety

| Structure | Class |
|---|---|
| 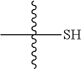 [SMILES: *S] | Sulfhydryl |
| 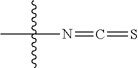 [SMILES: *N=C=S] | Isothiocyanate |
| 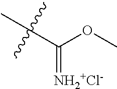 | Imidoester |
| 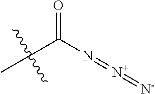 [SMILES: *C(=O)N=[N+]=[N-]] | Acylazide |
| 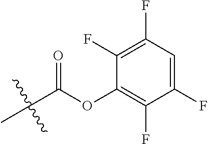 [SMILES: *C(=O)Oc1c(F)c(F)cc(F)c1F] | Activated ester |

TABLE 1-2

| Structure | Class |
|---|---|
| 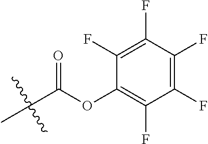 [SMILES: *C(=O)Oc1c(F)c(F)c(F)c(F)c1F] | Activated ester |
| 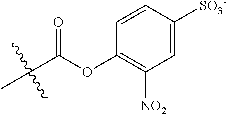 | Activated ester |
| 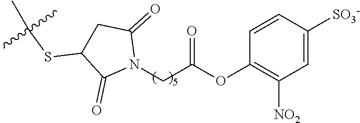 | Activated ester |
| 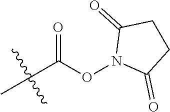 [SMILES: *C(=O)ON1C(=O)CCC1=O] | Activated ester |
| 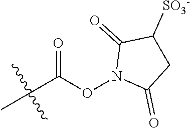 | Activated ester |
| 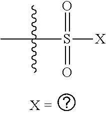 | Sulfonyl halide |
| 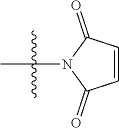 [SMILES: *N1C(=O)C=CC1=O] | Maleimide |

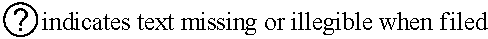

TABLE 1-3

| Structure | Class |
|---|---|
| 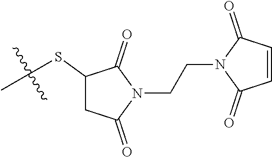 [SMILES: *SC1CC(=O)N(CCN2C(=O)C=CC2=O)C1=O] | Maleimide |
| 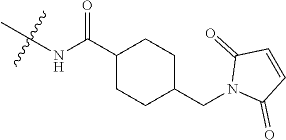 [SMILES: *NC(=O)C1CCC(CN2C(=O)C=CC2=O)CC1] | Maleimide |
| 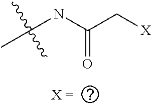 | α-Haloamide |
| 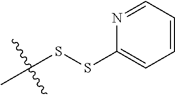 [SMILES: *SSc1ccccn1] | Disulfide |
| 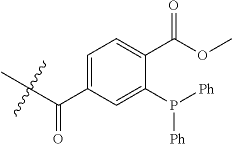 [SMILES: *C(=O)c1ccc(C(=O)OC)c(P(c2ccccc2)c2ccccc2)c1] | Phosphine |
| 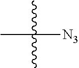 [SMILES: *N=[N+]=[N-]] | Azide |
| 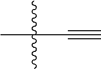 [SMILES: *C#C] | Alkyne |
| 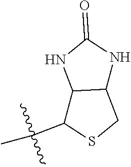 [SMILES: *C1SCC2NC(=O)NC21] | Biotin |
| 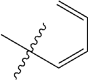 [SMILES: */C=C\C=C] | Diene |
| 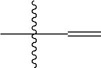 [SMILES: *C=C] | Alkene/dienophile |

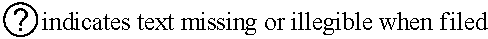

Regarding the compound having the structure (I), m may be independently at each occurrence an integer of 1 to 10, and particularly an integer of 1 to 5.

Regarding the compound having the structure (I), n may be an integer of 1 to 10.

Regarding the compound having the structure (I), M may be independently at each occurrence, pyrene, perylene, perylene monoimide, or 6-FAM, or a derivative thereof.

Regarding the compound having the structure (I), M may have independently at each occurrence one of the following structures:

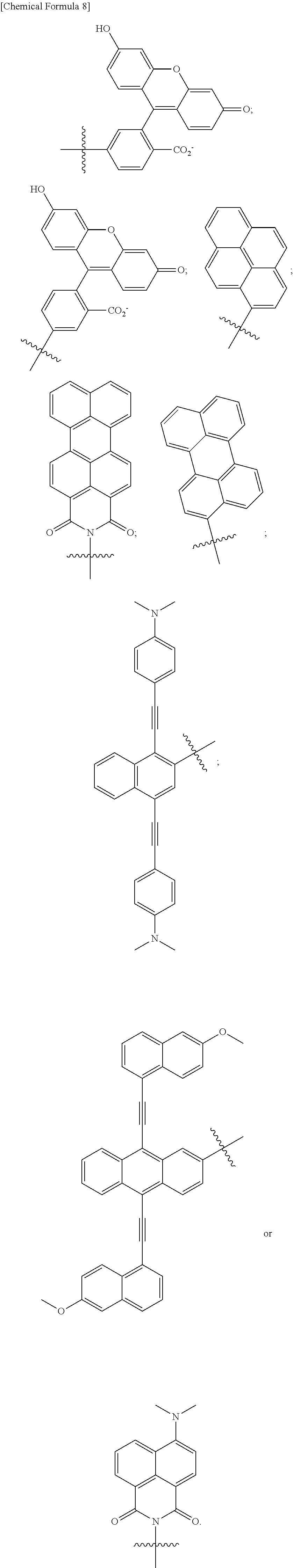

The compound having the structure (I) may be, for example, any compound selected from the compounds described in Table 2 of International Publication No. 2017/177065.

The bioparticle binding part 133 may be an antigen binding substance that binds to an antigen on the surface of the bioparticle P, or a molecule binding substance that binds to a molecule forming the surface membrane of the bioparticle P. The configuration of the bioparticle binding part 133 may be appropriately selected or designed by those skilled in the art according to the type of the bioparticle P.

The antigen binding substance may contain a substance selected from the group consisting of an antibody, an antibody fragment, an aptamer, and a molecularly imprinted polymer. The antibody or the antibody fragment may be, for example, an antibody or an antibody fragment that binds to a component (particularly, a surface antigen) present on the surface of a bioparticle such as a cell. The aptamer may be a nucleic acid aptamer or a peptide aptamer. The aptamer and the molecularly imprinted polymer may also bind to, for example, a component (particularly, a surface antigen) present on the surface of a bioparticle such as a cell.

The molecule binding substance is, for example, a compound having an oleyl group or a cholesteryl group. These groups can non-specifically bind to a molecule forming a surface membrane of the bioparticle P (for example, a cell). The oleyl group and the cholesteryl group may bind to, for example, a bioparticle including a lipid bilayer membrane, such as a cell. Examples of these compounds are as described above with reference to FIG. 3B in the item (3-1-1) surface preparation step”.

The capturing substance binding step S113 may include an incubation step for binding the bioparticle and the first capturing substance. Incubation conditions such as incubation time and temperature may be determined according to the types of the bioparticle and the first capturing substance to be used.

After the capturing substance binding step S113 is executed, a removal step of removing unnecessary substances such as first capturing substances that have not bound to the particle capturing substance 120 may be performed. The removal step may include washing the surface 110 with a liquid, for example, a buffer.

In the flowchart of FIG. 1B, the capturing substance binding step S113 is described to be performed after the surface capturing step S112 and before the cleavage step S114, but the timing at which the capturing substance binding step S113 is performed is not limited thereto.

The capturing substance binding step S114 may be performed before the surface capturing step S112, or may be performed while the surface capturing step S112 is performed.

For example, the bioparticle-containing sample and the first capturing substance are mixed, and the first capturing substance is bound to the bioparticle contained in the sample. Then, the bioparticle to which the first capturing substance has been bound in advance may be captured on the surface 110 using the bioparticle-containing sample to which the binding has been performed in the surface capturing step S112.

Alternatively, the bioparticle-containing sample and the first capturing substance are applied to the surface 110, and the first capturing substance may be bound to each bioparticle while the bioparticle contained in the sample is captured on the surface 110.

Alternatively, the bioparticle-containing sample is applied to the surface 110, and the bioparticle contained in the sample is captured on the surface 110. Then, after the capturing is completed, the first capturing substance is applied to the surface 110, and the first substance may be bound to each bioparticle.

### Modified Example 1: Embodiment in which Secreted Substance Binding Part Contains a Plurality of Secreted Substance Binding Substances

The secreted substance binding part may contain one secreted substance binding substance as described with reference to FIG. 4, or may contain a plurality of the same or different secreted substance binding substances. The first capturing substance in a case where the secreted substance binding part contains a plurality of the same or different secreted substance binding substances will be described with reference to FIG. 17.

FIG. 17 is a schematic view illustrating an example of a state in which the first capturing substance is bound to the bioparticle (cell) P. A first capturing substance 330 illustrated in FIG. 17 includes a secreted substance binding part 331, a crosslinking part 332, and a bioparticle binding part 333.

The secreted substance binding part 331 contains four secreted substance binding substances (antibodies) 331-1, 331-2, 331-3, and 331-4. These four antibodies may be the same or different from each other. For example, the four antibodies may be configured to capture the same secreted substance (such as a cytokine), or may be configured to capture different secreted substances. As described above, the plurality of secreted substance binding substances contained in the secreted substance binding part may be the same or different from each other. For example, the plurality of secreted substance binding substances may be antibodies that bind to different antigens. Furthermore, the plurality of secreted substance binding substances is not necessarily an antibody, and may be, for example, any of an antibody fragment, an aptamer, and a molecularly imprinted polymer. Furthermore, the plurality of secreted substance binding substances contained in the secreted substance binding part may have the same secreted substance binding properties or different secreted substance binding properties.

The crosslinking part 332 binds to the plurality of secreted substance binding substances 333-1 to 333-4, and also binds to the bioparticle binding part 333. The crosslinking part 332 having such a plurality of binding sites may be a compound having the structure (I) described above as an example of the crosslinking part 132, but is not limited thereto. The crosslinking part 132 may be selected from compounds having a plurality of binding sites, known in the art, such as a compound having the structure (I).

The bioparticle binding part 333 is illustrated as an antibody in FIG. 17, but may be an antigen binding substance or a molecule binding substance other than the antibody, as described for the bioparticle binding part 133.

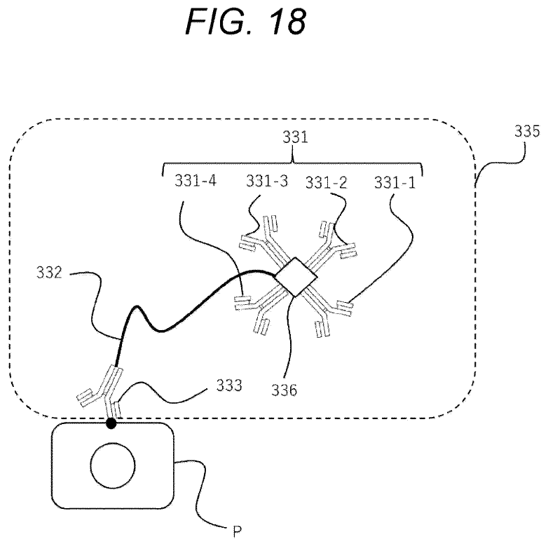

Furthermore, the plurality of secreted substance binding substances is not necessarily bound to one linear compound as illustrated in FIG. 17, and may be bound to a substance bound to the crosslinking part 332. An example of this is illustrated in FIG. 18. In a first capturing substance 335 illustrated in FIG. 18, a granular substance 336 is bound to one end of the crosslinking part 332, and the plurality of secreted substance binding substances 331-1 to 331-4 is bound to the granular substance 336.

### Modified Example 2: Embodiment in which Bioparticle Binding Part is Multispecific Antibody

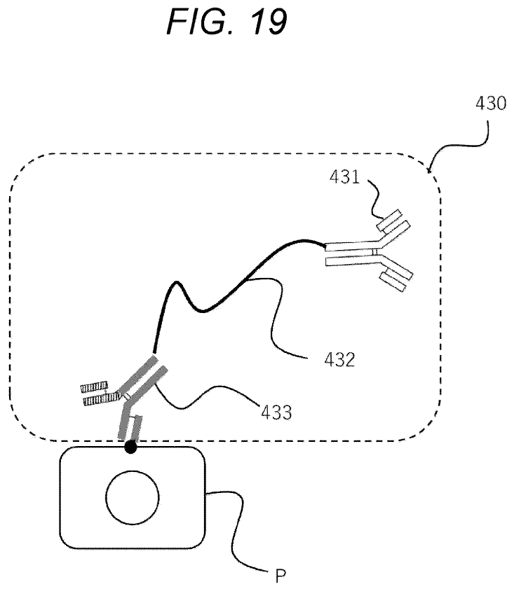

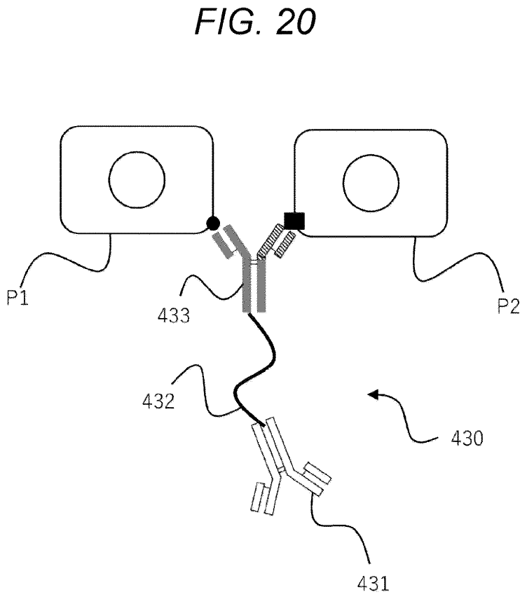

The bioparticle binding part may be an antigen binding substance that binds to an antigen on the surface of the bioparticle P. Furthermore, the antigen binding substance may be a multispecific antibody, particularly a bi-specific antibody or a tri-specific antibody. This modified example will be described with reference to FIG. 19.

FIG. 19 is a schematic view illustrating an example of a state in which the first capturing substance is bound to the bioparticle (cell) P. A first capturing substance 430 illustrated in FIG. 19 includes a secreted substance binding part 431, a crosslinking part 432, and a bioparticle binding part 433.

The secreted substance binding part 431 contains one secreted substance binding substance (antibody). Note that the secreted substance binding substance is not necessarily an antibody, and may be, for example, any of an antibody fragment, an aptamer, and a molecularly imprinted polymer.

The crosslinking part 432 may be a compound having the structure (I) described above as an example of the crosslinking part 132, but is not limited thereto.

As illustrated in FIG. 19, the bioparticle binding part 433 may be a bi-specific antibody. The bi-specific antibody may be, for example, an antibody that binds (specifically) to a surface antigen of the cell P and binds (specifically) to a cell other than the cell P.

FIG. 20 illustrates a state in which two cells P1 and P2 are captured by one first capturing substance 430. The bioparticle binding part 433 of the first capturing substance 430 is a bi-specific antibody, and binds to a surface antigen (black circle) of the cell P1 and a surface antigen (black square) of the cell P2. The surface antigen of the cell P1 is different from the surface antigen of the cell P2, and two antigens different from each other are captured by one bioparticle binding part (antibody) 433.

### Modified Example 3: First Capturing Substance Containing Antibody that Binds to Two or More Bioparticles

In the modified example of the present disclosure, the first capturing substance may contain an antibody that binds to the surfaces of two or more same or different type of bioparticles (particularly cells), and more particularly may contain an antibody that binds to the surfaces of two or more different types of bioparticles. The antibody may be an antibody that binds to two or more different antigens. The antibody may be, for example, a so-called multispecific antibody, more specifically, a bi-specific antibody or a tri-specific antibody. In the present modified example, the first capturing substance may contain the antibody separately from the secreted substance binding part and the bioparticle binding part.

In the modified example, for example, two or more cells, particularly two or more cells different from each other are captured by the antibody. That is, in the first capturing step S102, the antibody contained in the first capturing substance captures two or more cells, and these cells are retained at extremely close positions from each other. Therefore, intercellular interaction between the two or more cells can be intentionally caused. For example, the intercellular interaction may be, for example, an interaction between one immune cell and one tumor cell, an interaction between one immune cell and another immune cell, or an interaction among one immune cell, another immune cell, and one tumor cell. That is, the antibody may be an antibody that captures two or more same or different immune cells, or an antibody that captures one or more immune cells and one or more tumor cells. When the first capturing substance contains the antibody, the intercellular interaction can be more efficiently analyzed. This is very useful in research and development of an antibody drug or a cell therapeutic agent.

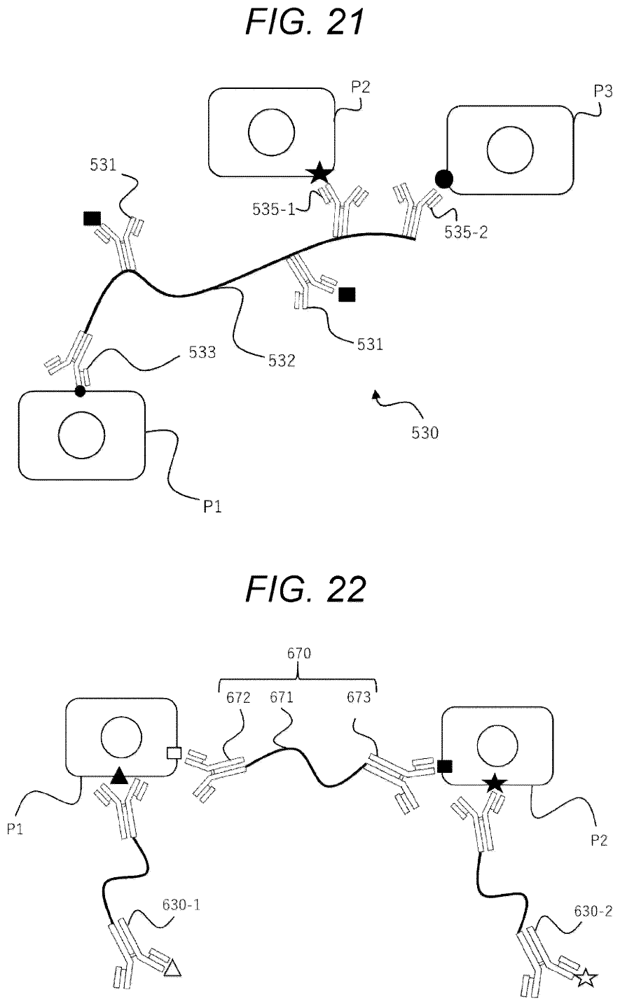

This modified example will be described with reference to FIG. 21. As illustrated in FIG. 21, a first capturing substance 530 includes a secreted substance binding part 531, a crosslinking part 532, and a bioparticle binding part 533 that binds to the cell P1. The first capturing substance 530 further includes antibodies 535-1 and 535-2 that bind to the surfaces of cells. The antibody 535-1 binds to the surface antigen (black star) of the cell P2. The antibody 535-2 binds to the surface antigen (black circles) of a cell P3. When the antibodies 535-1 and 535-2 bind to the cells P2 and P3, respectively, the cells P2 and P3 are kept close to each other. When the cells P2 and P3 are kept close to each other, an interaction occurs between these cells. By the interaction, for example, secreted substances (black square marks) are released from these cells. The secreted substance is captured by the secreted substance binding part 531. In this way, the intercellular interaction can be analyzed.

In this modified example, the secreted substance binding part of the first capturing substance may be configured to bind to the secreted substance generated by the intercellular interaction. Furthermore, in this modified example, the second secreted substance binding part of the second capturing substance described later may also be configured to bind to the secreted substance at a site different from the site to which the secreted substance binding part has been bound.

### Modified Example 4: Crosslinking of Two or More Bioparticles

In still another modified example of the present disclosure, two or more bioparticles may be crosslinked in the first capturing step. By the crosslinking, for example, a state in which two or more cells are present close to each other is maintained, and this can cause the intercellular interaction.

In this modified example, a crosslinking substance similar to the first capturing substance may be used in order to perform the crosslinking. The crosslinking substance will be described with reference to FIG. 22. A crosslinking substance 670 illustrated in FIG. 22 includes two bioparticle binding parts 672 and 673 and a crosslinking part 671. The bioparticle binding parts 672 and 673 may be similar to the other bioparticle binding parts described above. The crosslinking part 671 may be similar to the crosslinking part described above. The bioparticle binding parts 672 and 673 bind to the surface antigens of the cells P2 and P3, respectively. Therefore, a state in which the cells P1 and P2 are present close to each other is maintained by the crosslinking substance 670. As a result, an interaction between the cells P1 and P2 occurs. The secreted substances generated by the interaction are captured by, for example, the first capturing substances 630-1 and 630-2 according to the present disclosure.

Since the bioparticle binding parts 672 and 673 are substances that bind to the bioparticle in a specific binding manner, such as an antibody, a plurality of specific bioparticles (cells) can be crosslinked. As a result, an interaction between specific cells can be analyzed.

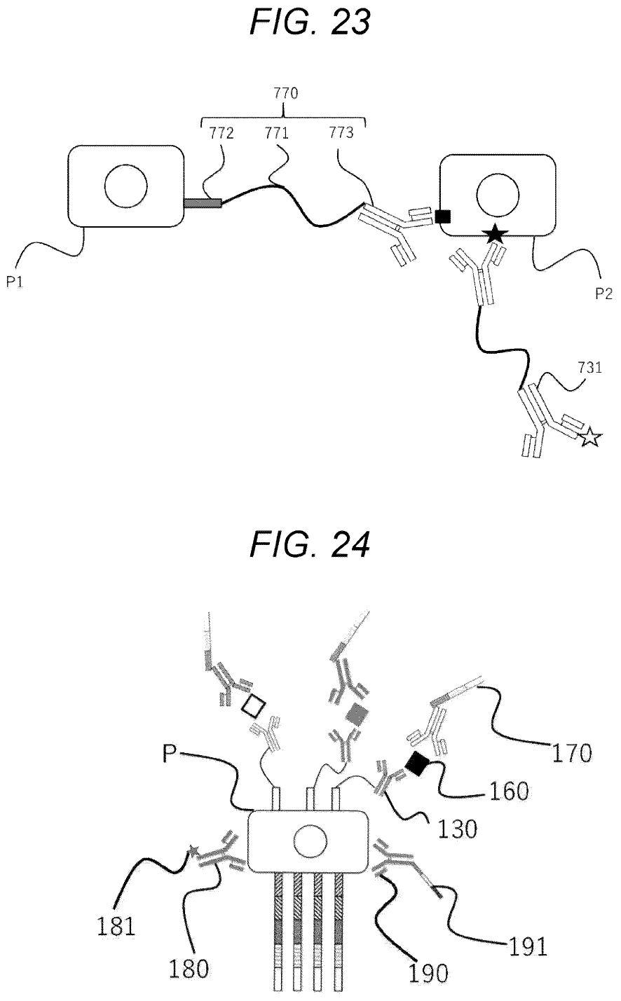

The crosslinking substance may bind in a non-specific manner. For example, a crosslinking substance 770 illustrated in FIG. 23 includes two bioparticle binding parts 772 and 773 and a crosslinking part 771. The bioparticle binding part 772 is a substance that binds to various cells in a non-specific manner, and is, for example, the compound having an oleyl group or a cholesteryl group described above. The bioparticle binding part 773 is an antibody that binds to a cell in a specific manner. The crosslinking part 771 may be similar to the crosslinking part described above. By the crosslinking substance 770, a specific cell is crosslinked with various cells. This makes it possible to analyze an interaction between a specific cell and various cells.

### (3-1-4) Cleavage Step

In the cleavage step S114, the linker 126 is cleaved, and the bioparticle captured in the surface capturing step S112 is released from the surface 110. Preferably, in the cleavage step S114, the captured state of the bioparticle P captured by the particle capturing part 121 is maintained. The captured state may be maintained until the environmental transition of the bioparticle in the environment transition step S104 described later is completed, or may be maintained, for example, until the disruption of the bioparticle in the disruption step S105 described later is completed.

The cleavage may be performed over the entire region of the surface 110, or may be performed on a partial region of the surface 110. In the latter case, the partial region may be selected on the basis of, for example, the detection result of the detection step described below.

Furthermore, the cleavage may be executed so as to release the entire bioparticle captured on the surface 110 from the surface 110, or may be executed so as to release a part of the bioparticle captured on the surface 110 from the surface 110. In the latter case, the part of the bioparticle may be selected on the basis of, for example, a detection result of the detection step described below.

For example, the bioparticle to be released from the surface 110 may be selected on the basis of a label of the bioparticle P, a label of the particle capturing substance 120, or a label of the first capturing substance 130.

The label of the bioparticle P may be, for example, a fluorescent dye constituting a fluorochrome-labeled antibody, or a label (particularly, a fluorescent dye) present inside the bioparticle.

The label of the particle capturing substance 120 is, for example, a fluorescent dye. A part of the nucleic acid contained in the particle capturing substance 120 may be a nucleic acid labeled with a fluorescent dye. Alternatively, the antibody contained in the particle capturing substance 120 may be labeled with a fluorescent dye.

The label of the first capturing substance 130 is also, for example, a fluorescent dye. A part of the nucleic acid contained in the first capturing substance 130 may be a nucleic acid labeled with a fluorescent dye. Alternatively, the antibody contained in the first capturing substance 130 may be labeled with a fluorescent dye.

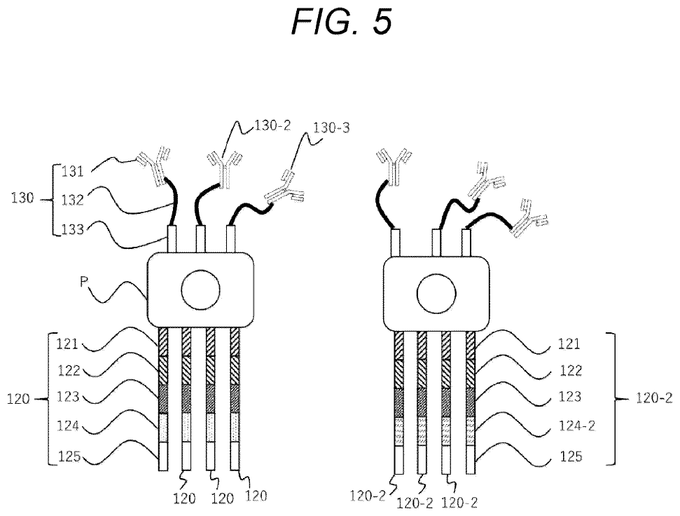

The bioparticle included in the bioparticle population obtained in the cleavage step S114 will be described with reference to FIG. 5. FIG. 5 is a schematic view of the bioparticle.

As illustrated on the left side of FIG. 5, a plurality of first capturing substances 130, 130-2, and 130-3 for capturing the secreted substance and a plurality of particle capturing substances 120 are bound to the bioparticle P. In the bioparticles included in the bioparticle population, different particle identifiers may be bound for each particle. The particle identifier 124 is bound to the bioparticle illustrated on the left of FIG. 5, but a particle identifier 124-2 different from the particle identifier 124 is bound to the bioparticle illustrated on the right of FIG. 5. The difference in the particle identifier may be, for example, a difference in the base sequence constituting the particle identifier. As described above, the bioparticles included in the bioparticle population obtained in the preparation step may have different particle identifiers. Furthermore, a plurality of particle identifiers bound to one bioparticle may be the same. Such a bioparticle population is suitable for executing single cell analysis in the analysis step described later.

In one embodiment of the present disclosure, the cleavage step S114 may include a detection step of detecting light generated from the bioparticle or light from a substance bound to the bioparticle, and a linker cleavage step of cleaving the linker on the basis of the detection result in the detection step to release the bioparticle from the surface 111. As a result, for example, the bioparticle to be released from the surface 110 can be selected according to the detection result. As a result, unintended bioparticles can be excluded from the target in the analysis step described later, and the efficiency of analysis can be improved.

In another embodiment of the present disclosure, in the cleavage step S114, the linker cleavage step may be executed without executing the detection step. By omitting the detection step, the number of steps in the analysis method of the present disclosure can be reduced.

Hereinafter, the detection step and the linker cleavage step will be described.

### (3-1-4-1) Detection Step

The cleavage step S114 may include a detection step of detecting any one or two or more of light derived from the bioparticle (for example, scattered light and/or intrinsic fluorescence); light derived from the target capturing molecule (for example, fluorescence); light derived from the antibody bound to the bioparticle (for example, fluorescence); the morphology of the bioparticle (for example, the morphology (the morphology that is characterized by an image acquired in the bright field, the phase contrast, or the dark field and that is characterized by image processing in the bright field, the phase contrast, or the dark field, in particular, the morphology acquired by morphology processing) or a state in which two or more bioparticles (such as cells) are bound to each other); and the feature of the bioparticle predicted from the morphological information of the bioparticle (for example, cell type or cell state (living cell or dead cell). These light, morphology, feature, and the like may be detected by, for example, an observation device including an objective lens, particularly a microscope device. These light, morphology, and feature may be detected, for example, by an imaging element or a photodetector. On the basis of the detection result of light, morphology, feature, and the like in the detection step, target capturing molecules to be cleaved in the linker cleavage step described later may be selected, or bioparticles to be released from the surface 110 in the cleavage step S114 may be selected. For example, the imaging element acquires an image of the surface 110 or an image of the bioparticles captured on the surface 110, and bioparticles to be released may be selected on the basis of the acquired image.

### (3-1-4-2) Linker Cleavage Step

The cleavage step S114 includes a linker cleavage step of cleaving the linker 126. By the cleavage of the linker 126, the bioparticle to which the first capturing substance and the particle capturing substance are bound is released from the surface 110. When the linker 1 of the particle capturing substance 120 is cleaved, for example, as illustrated in c of FIG. 2A, the particle capturing substance 120 is released from the surface 110, and along with this releasing, the bioparticle is also released from the surface 110.

In the cleavage step S114, the linker may be cleaved by, for example, stimulation such as chemical stimulation or light stimulation. Light stimulation is particularly suitable for selectively stimulating a specific narrow range.

Stimulation in the cleavage step S114 may be performed by a stimulus application device. The drive of the stimulus application device may be controlled by, for example, an information processing device such as a general-purpose computer. For example, the information processing device may drive the stimulus application device, to cause the stimulus application device to selectively apply a stimulus to a position of the bioparticle to be released. An example of a stimulus application device that may be adopted will be described below.

In order to selectively apply a light stimulus to the position of the cell, a light irradiation device may be used as a stimulus application device. The light irradiation device may be, for example, a digital micromirror device (DMD) or a liquid crystal display device. A selected position on the surface 110 can be irradiated with light by the micromirror constituting the DMD. The liquid crystal display device may be, for example, a reflective type liquid crystal display, and a specific example thereof include SXRD (Sony Corporation). By controlling the liquid crystal of the liquid crystal display device, a selected position of the surface 110 can be irradiated with light.

Furthermore, a liquid crystal shutter or a spatial light modulator may be used to selectively apply a light stimulus to the position of the cell. These devices can also apply a light stimulus to the selected position.

The wavelength of the irradiation light may be appropriately selected by those skilled in the art according to the type of the linker contained in the particle capturing substance.

The chemical stimulus may be applied by bringing a reagent for cleaving the linker 126 into contact with the surface 110. As described above, the reagent may be determined according to the type of the linker 126.

For example, in a case where the linker 126 contains a disulfide bond, the reagent may be a reducing agent capable of cleaving the bond. The reagent may be, for example, tris(2-carboxyethyl) phosphine (TCEP), dithiothreitol (DTT), or 2-mercaptoethanol. For example, in a case where TCEP is used, the reaction is performed using 50 mM of TCEP for about 15 minutes, for example.

For example, in a case where the linker 126 is a nucleic acid including a restriction endonuclease recognition sequence, the reagent may be a restriction endonuclease corresponding to each restriction endonuclease recognition sequence. 1 U of the restriction endonuclease activity is the amount of enzyme that completely degrades 1 μg of λDNA per hour at 37° C. in principle in 50 μl of each enzyme reaction solution. The amount of enzyme may be adjusted according to the amount of the restriction endonuclease recognition sequence.

The at least one bioparticle released by the cleavage in the cleavage step S114 may be recovered in, for example, a liquid such as a buffer or a medium. The liquid may be, for example, a hydrophilic liquid. The bioparticle-containing liquid obtained by the recovering may be used in the environment transition step S104 described later. In order to recover the released bioparticle, a fluid force generated by causing a liquid such as a buffer to flow may be used. Alternatively, the bioparticle may be floated in the liquid by vibration, or the bioparticle may be floated in the liquid by using gravity or the like. The vibration may be, for example, vibration transmitted through the substrate 100 or vibration transmitted through the liquid containing the bioparticle. In addition, the substrate 110 may be moved such that the surface 110 faces the direction of gravity in order to float the bioparticle in the liquid by the gravity.

### (3-2) First Capturing Step

The first capturing step S102 includes a treatment step of performing a treatment of placing the bioparticle population prepared in the preparation step S101 under a predetermined condition. The treatment step may be performed while a population state of the bioparticle population is maintained. A secreted substance generated by placing the bioparticle population under a predetermined condition and the first capturing substance bound to each bioparticle included in the bioparticle population are bound. The first capturing step S102 may be performed while a state in which the first capturing substance is bound to the bioparticle is maintained.

The predetermined condition may be a condition under which the reactivity of the bioparticle population is analyzed, and may be appropriately selected by the user who executes the bioparticle analysis method of the present disclosure. The predetermined condition may be, for example, a condition under which a secreted substance is generated, or a condition under which whether or not a secreted substance is generated is analyzed. The generated secreted substance is captured by the first capturing substance.

More specifically, the predetermined condition is an incubation environment of the bioparticle population (particularly, a cell population), and is for example, an environment in a medium or a buffer solution. In the first capturing step S102, a secreted substance generated by placing the bioparticle population under the environment is captured by the first capturing substance.

The incubation environment may contain, for example, a biomaterial or a non-biomaterial. In the present disclosure, the reactivity of the bioparticle population under an environment in which the material exists may be analyzed. The material may be, for example, a diseased tissue, a diseased cell, a microorganism (bacteria, fungus, or virus), a substance that causes disease or increases the risk of disease (for example, a carcinogen, amyloid B, prion, and the like), a drug, a toxic substance, or a heterologous tissue. The non-biomaterial may be, for example, a drug or a toxic substance. The diseased tissue may be, for example, a tumor tissue, and in particular, may be a cancer tissue or a sarcoma tissue. The diseased cell may be, for example, a tumor cell, and in particular, may be a cancer cell, a sarcoma cell, or a malignant lymphoma cell.

The secreted substance may be a secreted substance secreted from a bioparticle included in the bioparticle population, or may be a secreted substance secreted from a material used for constituting the predetermined condition. The secreted substance may be, for example, a secreted substance secreted from a diseased tissue, a diseased cell, a microorganism, or a heterologous tissue.

The bioparticle may be a cell as described above, and the secreted substance secreted from the bioparticle may be a secreted substance secreted from a cell. For example, the secreted substance may be a substance secreted by an immune cell, and may be, for example, any one or more selected from cytokines, hormones, antibodies, and exosomes, but is not limited thereto. The secreted substance may be a substance secreted by a nerve cell, a muscle cell, a skin cell, or a glandular cell. The secreted substance may be an exosome.

The secreted substance may be, for example, a protein, a peptide, an exosome, or other biomolecules from the viewpoint of the material. The secreted substance may be, for example, an exosome, a cytokine, a hormone, or a neurotransmitter from the viewpoint of the type of cell.

Furthermore, the secreted substance generated by placing the bioparticle population under a predetermined condition is not limited to the substance secreted from the cell included in the bioparticle population. The secreted substance may be, for example, a secreted substance secreted from a material constituting the predetermined condition. The material may be a material contained in the incubation environment described above. The material may be a biological tissue, a cell (particularly, a diseased cell), a microorganism, or a heterologous tissue, particularly a diseased tissue, and more particularly a tumor tissue or a neurodegenerative tissue. The cell is, for example, a diseased cell, particularly a tumor cell.

A specific example of the first capturing step S102 will be described with reference to FIG. 2B.

In order to execute the first capturing step S102, for example, an incubation environment is prepared as a predetermined condition. The incubation environment may be an environment in a container 140 as illustrated in d of FIG. 2B. The container 140 is, for example, a petri dish, a well plate, a tube, or the like, but is not limited thereto. The container 140 contains, for example, an incubation medium such as a medium or a buffer solution. The container 140 further contains a diseased cell group (tumor cell) 145 as a material constituting the incubation environment. The diseased cell group 145 may include one type or two or more types of cells. In d of FIG. 2B, the diseased cell group 145 includes two types of cells (cells 145a and 145b).

As illustrated in d of FIG. 2B, the bioparticle population prepared in the preparation step is placed in the container 140. Then, the bioparticle population is incubated in the container. The time and/or temperature of the incubation may be appropriately selected by those skilled in the art so that secreted substances are generated.

Secreted substances are generated in the container 140 by the incubation. As described above, the secreted substance may be a substance generated from the bioparticle included in the bioparticle population, a substance generated from the material constituting the incubation environment (the diseased cell group in FIG. 2B), or both of these substances.

e of FIG. 2B illustrates that secreted substances 160, 161 and 162 have been generated. As illustrated in the drawing, these generated secreted substances are captured by the first capturing substance 130. In e of FIG. 2B, a plurality of types of secreted substances different from each other is generated, but one type of secreted substance may be generated.

### (3-3) Second Capturing Step

In the second capturing step S103, the secreted substance bound to the first capturing substance is bound to a second capturing substance for capturing the secreted substance. Thereby, a conjugate of the first capturing substance, the secreted substance, and the second capturing substance is formed. The second capturing substance is preferably configured to bind to a site different from the site to which the first capturing substance binds. The second capturing step S103 may be performed while a state in which the first capturing substance is bound to the bioparticle is maintained.

In a preferred embodiment of the present disclosure, both the first capturing step S102 and the second capturing step S103 are performed while a state in which the first capturing substance is bound to the bioparticle is maintained. As a result, a sandwich structure described later is formed on the bioparticle. Forming such a structure is useful, for example, for analyzing interactions between bioparticles included in the bioparticle population.

The second capturing step S103 may be executed in an incubation environment in which the first capturing step S102 has been performed, or may be executed in an environment different from the incubation environment. The second capturing step S103 is preferably executed in another environment of the latter, from the viewpoint of efficiency of conjugate formation. For example, after completion of the first capturing step S102, the bioparticle population including the bioparticle having the first capturing substance to which the secreted substance has been bound is recovered from the incubation environment. Then, the recovered bioparticle population is transferred to an incubation environment for executing the second capturing step S103 (hereinafter, also referred to as “second incubation environment”). The second incubation environment may be an environment that allows binding between the second secreted substance binding part described later and the secreted substance, and may be an environment in the container. The container is, for example, a petri dish, a well plate, a tube, or the like, but is not limited thereto. The container may contain, for example, an incubation medium such as a medium or a buffer solution.

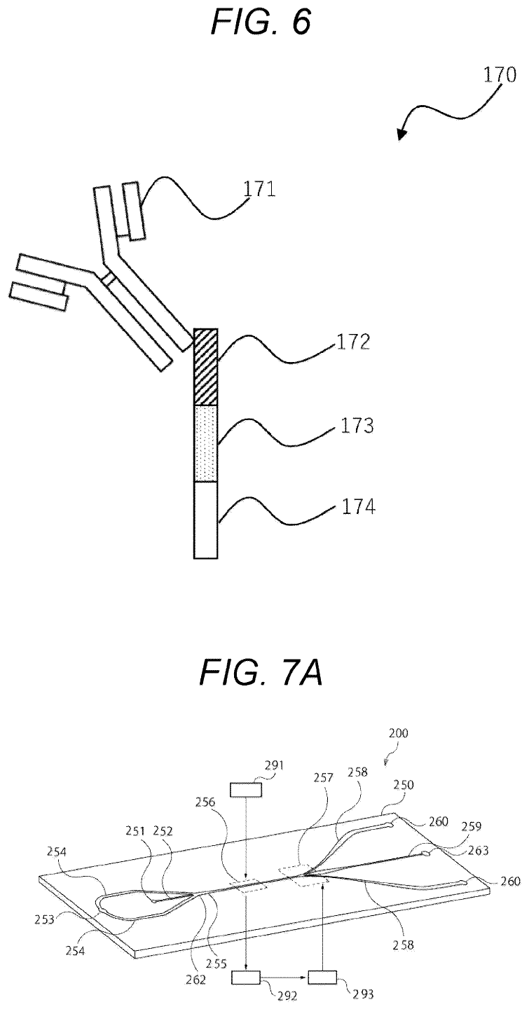

A configuration example of the second capturing substance will be described with reference to FIG. 6. As illustrated in FIG. 6, a second capturing substance 170 includes a second secreted substance binding part 171, a second recovered substance amplification part 172, a capturing substance identifier 173, and a poly A sequence 174. As described later, the second capturing substance is, for example, a complex of a nucleic acid and a protein, and can be appropriately produced by those skilled in the art.

The second secreted substance binding part 171 may be appropriately designed or produced by those skilled in the art according to the secreted substance to be bound to the second secreted substance binding part 171. For example, the second secreted substance binding part 171 may be, for example, a substance selected from the group consisting of an antibody, an antibody fragment, an aptamer, and a molecularly imprinted polymer, and is particularly an antibody or an antibody fragment. Note that, in FIG. 6, an antibody is illustrated as the second secreted substance binding part 171. The binding of the second secreted substance binding part 171 may be specific or non-specific, and is particularly specific.

The second secreted substance binding part 171 is configured to bind to the secreted substance to which the first capturing substance 130 binds, and particularly configured to bind to a portion different from the portion to which the first capturing substance 130 binds in the secreted substance to which the first capturing substance 130 binds.

In the second capturing step S103, a state in which the second capturing substance 170 is bound to the secreted substance to which the first capturing substance 130 is bound is formed. A state in which two different antibodies bind to one substance is also called, for example, a sandwich structure. In I the second capturing step S103, such a sandwich structure may be formed. More specifically, a structure in which the secreted substance binding part 131 (for example, an antibody) included in the first capturing substance 130 and the second secreted substance binding part 171 (for example, an antibody) included in the second capturing substance 170 are bound to one secreted substance may be formed. Note that one type of the second secreted substance binding part may be bound to one secreted substance, or two or more types of the second secreted substances may be bound to one secreted substance.

The second recovered substance amplification part 172 contains, for example, a primer for nucleic acid amplification and/or a promoter for nucleic acid transcription. Particularly, the second recovered substance amplification part 172 may contain a nucleic acid having a primer sequence used for amplification of a nucleic acid, or a promoter sequence used for transcription of a nucleic acid in the analysis step described later. The nucleic acid may be DNA or RNA, and is particularly DNA. The second recovered substance amplification part 172 may have both a primer sequence and a promoter sequence. The primer sequence may be, for example, a PCR handle. The promoter sequence may be, for example, a T7 promoter sequence.

The capturing substance identifier 173 is used for identifying or specifying the second capturing substance including the capturing substance identifier, or the second secreted substance binding part. The capturing substance identifier 173 contains, for example, a nucleic acid having a barcode sequence. The nucleic acid may be particularly DNA or RNA, and more particularly DNA. The barcode sequence may be used, for example, for specifying the second capturing substance bound to the secreted substance, or the second secreted substance binding part. For the specifying, the barcode sequence may be associated with the second capturing substance including the barcode sequence, or the second secreted substance binding part. Therefore, the barcode sequence may be associated with the second capturing substance or the second secreted substance binding part. For example, the sequence information of the barcode sequence may be associated with the type of the second capturing substance or the second secreted substance binding part. The barcode sequence may be associated with the second capturing substance or the second secreted substance binding part, for example, on a one-to-one basis.

As described above, a capturing substance identifier for identifying the second capturing substance may be bound to the second capturing substance 173. As a result, the capturing substance bound to the bioparticle in the analysis step described later can be specified.

The poly A sequence 174 can stabilize an amplification product of the barcode sequence when the barcode sequence is read in the analysis step described later.

A specific example of the second capturing step S103 will be described with reference to FIG. 2C.

In order to execute the second capturing step S103, an incubation environment in which the secreted substance captured by the first capturing substance 130 in the first capturing step S102 and the second capturing substance 170 are bound is prepared. The incubation environment may be an environment in a container 150 as illustrated in f of FIG. 2C. The container 150 is, for example, a petri dish, a well plate, a tube, or the like, but is not limited thereto. The container 150 contains, for example, an incubation medium such as a medium or a buffer solution.

As illustrated in f of FIG. 2C, the bioparticle population including the bioparticle P after the capturing treatment of the secreted substance in the first capturing step S102 and the second capturing substance 170 are placed in the container 150. Then, the bioparticle population is incubated in the container. The time and/or temperature of the incubation may be appropriately selected by those skilled in the art so that secreted substances are generated. By the incubation, the secreted substance 160 is captured by the second capturing substance 170. As a result, a state in which the secreted substance 160 is captured by the first capturing substance 130 and the second capturing substance 170 is formed.

### Modified Example 5: Use of Substance that Binds to Surface Molecule of Bioparticle

In the modified example of the present disclosure, in the second capturing step, a binding step of binding a surface molecule binding substance to a surface molecule of the bioparticle may be executed in addition to the capturing of the secreted substance by the second capturing substance. The surface molecule binding substance may be, for example, an antibody, an antibody fragment, an aptamer, or a molecularly imprinted polymer. For example, a fluorescent label or an identification substance may be bound to the surface molecule binding substance. The incubation is performed in a state in which the surface molecule binding substance is added to the incubation medium. As a result, in addition to the binding of the second capturing substance to the secreted substance, the surface molecule binding substance is bound to a surface molecule (particularly, a surface antigen) of the bioparticle.

The fluorescent label may be used, for example, for determining whether or not the bioparticle is to be isolated in a microspace in the isolation step described later. The identification substance is released from the surface of the bioparticle by disruption of the bioparticle in the disruption step described later, and then binds to, for example, a substance recovery part such as a poly T sequence to form a conjugate. The conjugate is used for specifying the surface molecule binding substance bound to the surface of the bioparticle in the analysis step described later.

This modified example will be described with reference to FIG. 24. To the bioparticle P illustrated in FIG. 24, a binding substance 180 labeled with a fluorescent label 181 and a binding substance 190 to which an identification substance 191 is bound are bound, in addition to the first capturing substance 130, the secreted substance 160, and the second capturing substance 170. In a case where the surface molecule binding substance is used, the state as illustrated in this drawing is formed in the second capturing step.

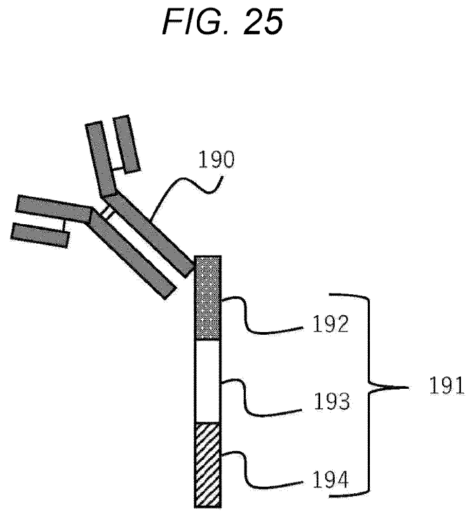

As the binding substance (for example, an antibody) 180 labeled with the fluorescent label 181, those known in the art may be adopted. The binding substance (for example, an antibody) 190 to which the identification substance 191 is bound will be described below with reference to FIG. 25.

As illustrated in FIG. 25, the identification substance 191 bound to the binding substance 190 includes a third recovered substance amplification part 192, a binding substance identifier 193, and a poly A sequence 194.

The description of the second recovered substance amplification part 172 described above applies to the third recovered substance amplification part 192.

The binding substance identifier 193 is used for identifying or specifying the binding substance 190. The binding substance identifier 193 contains, for example, a nucleic acid having a barcode sequence. The nucleic acid may be particularly DNA or RNA, and more particularly DNA. The barcode sequence may be used, for example, for specifying the binding substance 190. To specify the binding substance 190, the barcode sequence may be associated with the binding substance 190. For example, the sequence information of the barcode sequence may be associated with the type of the binding substance 190. The barcode sequence may be associated with the binding substance 190, for example, on a one-to-one basis.

The poly A sequence 194 can stabilize an amplification product of the barcode sequence when the barcode sequence is read in the analysis step described later.

### (3-4) Isolation Step

In the isolation step S104, the bioparticle included in the bioparticle population is isolated into a single particle. In the present specification, the term “isolate” may mean that, in the case of executing the disruption step described later, components contained in one bioparticle and substances bound to the one bioparticle (for example, the first capturing substance, the second capturing substance, the particle identifier, and the like) are brought into a state of not being mixed with components contained in another bioparticle and substances bound to the other bioparticle. For example, the term “isolate” may mean being isolated in a microspace as described later.

According to one embodiment of the present disclosure, in the isolation step S104, each bioparticle included in the bioparticle population is isolated in one microspace. The microspace may be a space in an emulsion particle or a space in a well. As described above, by executing the disruption step described later in the microspace, the components contained in one bioparticle, and the first capturing substance, the second capturing substance, and the particle identifier, which are bound to the one bioparticle, are not mixed with the components contained in the other bioparticle and the substances bound to the other particle.

Furthermore, by executing the isolation step S104, one bioparticle and substances (for example, the first capturing substance, the second capturing substance, the particle identifier, and the like) bound to the one bioparticle can be associated on a one-to-one basis.

In one embodiment of the present disclosure, the isolation step S104 may include a discrimination step of determining whether or not the bioparticle is isolated in the microspace, and a particle isolation step of isolating the bioparticle determined to be isolated in the discrimination step, in the microspace. This makes it possible to isolate only the target bioparticle in the microspace. Therefore, unintended bioparticles can be excluded from the target in the analysis step described later, and the efficiency of analysis can be improved, for example.

The discrimination may be performed, for example, on the basis of light generated from the bioparticle (for example, scattered light and/or intrinsic fluorescence) or light generated from a substance bound to the bioparticle, or a morphological image. The substance bound to the bioparticle may be, for example, a target capturing molecule, or may be an antibody (particularly, a fluorochrome-labeled antibody) bound to the bioparticle. The scattered light generated from the bioparticle may be, for example, forward scattered light and/or side scattered light. The doublet detection can be performed on the basis of the height and/or area value of the signal acquired by scattered light detection. Single cell determination by morphological image information is also possible. Whether or not the bioparticle is a dead cell can be determined from scattered light and/or a morphological image, or fluorescence after staining with a dead cell staining reagent, whereby the dead cell can be removed. In the present disclosure, the discrimination step may be performed immediately before the isolation step, whereby only the single cell to which the barcode is attached can be reliably isolated.

In another embodiment of the present disclosure, the particle isolation step may be executed without performing the discrimination step. By omitting the discrimination step, the number of steps in the analysis method of the present disclosure can be reduced.

Hereinafter, the discrimination step and the particle isolation step will be described.

### (3-4-1) Discrimination Step

In the discrimination step, it is determined whether or not each bioparticle included in the bioparticle population is isolated in a microspace. As described above, the discrimination may be performed on the basis of light generated from the bioparticle, or light generated from the substance bound to the bioparticle.

The discrimination step may include, for example, an irradiation step of irradiating the bioparticle with light, and a detection step of detecting light generated by the irradiation.

The irradiation step may be executed by, for example, a light irradiation unit that irradiates the bioparticle with light. The light irradiation unit may include, for example, a light source that emits light. Furthermore, the light irradiation unit may include an objective lens that condenses light on the bioparticle. The light source may be appropriately selected by those skilled in the art depending on a purpose of an analysis, and may be, for example, a laser diode, an SHG laser, a solid-state laser, a gas laser, a high brightness LED, or a halogen lamp, or may be a combination of two or more of them. The light irradiation unit may include other optical elements as needed in addition to the light source and the objective lens.

The detection step may be executed by, for example, a detection unit that detects light generated from the bioparticle or the substance bound to the bioparticle. In the detection unit, for example, light generated from the bioparticle or the substance bound to the bioparticle by light irradiation by the light irradiation unit may be, for example, scattered light and/or fluorescence. The detection unit may include, for example, a condensing lens that condenses light generated from the bioparticle, and a detector. As the detector, PMT, photodiode, CCD, CMOS and the like may be used, but the detector is not limited thereto. The detection unit may include another optical element as needed in addition to the condensing lens and the detector. The detection unit may further include, for example, a spectroscopic unit. Examples of optical components that form the spectroscopic unit may include a grating, a prism, and an optical filter, for example. The spectroscopic unit can detect, for example, light having a wavelength that should be detected separately from light having another wavelength. The detection unit may convert the detected light into an analog electric signal by photoelectric conversion. The detection unit may further convert the analog electric signal into a digital electric signal by AD conversion.

The discrimination step may be executed by a determination unit that performs determination processing as to whether or not the discrimination of the bioparticle is performed, on the basis of the light detected in the detection step. The processing by the determination unit may be realized by, for example, an information processing device such as a general-purpose computer, in particular, a processing unit included in the information processing device.

### (3-4-2) Particle Isolation Step

The isolation step includes a particle isolation step of isolating the bioparticle in a microspace. In the present disclosure, the microspace may mean a space having a dimension capable of accommodating one bioparticle as an analysis target. The dimension may be appropriately determined according to, for example, factors such as the size of the bioparticle. The microspace may also have a dimension capable of accommodating two or more bioparticles as an analysis target. This case may include, in addition to a case where one bioparticle is accommodated in one microspace, a case where two or more bioparticles are accommodated. The bioparticles in the microspace that accommodates two or more bioparticles may be excluded from the disruption target in the disruption step described later, or may be excluded from the analysis target in the analysis step described later.

Incidentally, in the disruption step described later, the conjugate of the first capturing substance, the secreted substance, and the second capturing substance, formed in the second capturing step is released from the bioparticle. Furthermore, in the disruption step described later, for example, a complex of a substance in the bioparticle and a particle identifier (in particular, a complex generated by binding mRNA in the bioparticle and the poly T sequence of the particle identifier) may be generated. In the present disclosure, each of the microspaces is preferably isolated from each other such that the conjugate (and optionally the complex) generated in one microspace does not migrate to another microspace. Examples of the microspace thus isolated include a space in an emulsion particle and a space in a well. That is, in a preferred embodiment of the present disclosure, the microspace may be a space in an emulsion particle or a space in a well. Hereinafter, examples of the particle isolation step in a case where the microspace is these spaces will be described.

### (3-4-2-1) Case of Space in Emulsion Particle

The emulsion particles may be produced using, for example, a microfluidic channel. The device includes, for example, a channel through which a first liquid forming an emulsion dispersoid flows, and a channel through which a second liquid forming a dispersion medium flows. The first liquid may contain bioparticles. The device further includes a region where the two liquids come into contact to form an emulsion.

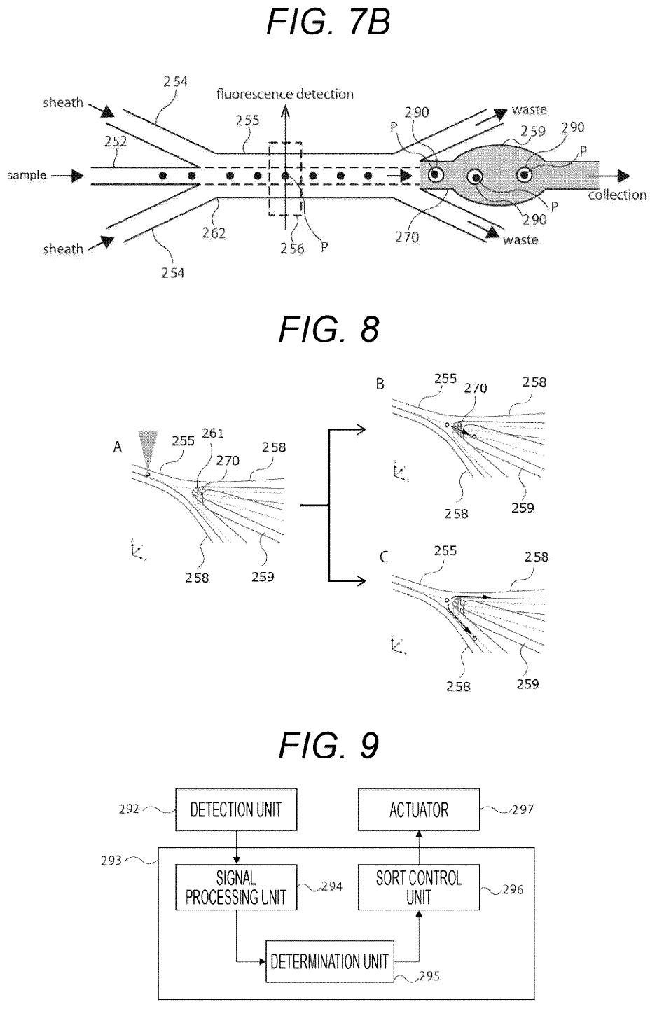

Hereinafter, an example of a device for efficiently forming an emulsion containing emulsion particles each including one bioparticle will be described with reference to FIGS. 7A and 7B. By the emulsion forming device, one bioparticle can be isolated in one emulsion particle with extremely high probability, and the number of empty emulsion particles can be reduced. Furthermore, the probability of isolating one bioparticle and one barcode sequence in one emulsion particle is also increased by the emulsion forming device.

FIG. 7A is an example of a microchip used for forming emulsion particles in the device. A microchip 250 illustrated in FIG. 7A includes a main channel 255 through which bioparticles flow, and a recovery channel 259 through which a recovery target particle among the bioparticles is recovered. The microchip 250 is provided with a particle sorting part 257. An enlarged view of the particle sorting part 257 is illustrated in FIG. 9. As illustrated in A of FIG. 8, the particle sorting part 257 includes a connection channel 270 that connects the main channel 255 and the recovery channel 259. A liquid supply channel 261 capable of supplying liquid to the connection channel 270 is connected to the connection channel 270. As described above, the microchip 250 has a channel structure including the main channel 255, the recovery channel 259, the connection channel 270, and the liquid supply channel 261.

FIG. 7B is a schematic view for illustrating formation of emulsion particles in the microchip 250 illustrated in FIG. 7A and isolation of bioparticles in the formed emulsion particles.

Furthermore, as illustrated in FIG. 7A, the microchip 250 forms a part of a bioparticle sorting device 200 including a light irradiation unit 291, a detection unit 292, and a control unit 293 in addition to the microchip. As illustrated in FIG. 9, the control unit 293 may include a signal processing unit 294, a determination unit 295, and a sort control unit 296. The bioparticle sorting device 200 is used as the emulsion forming device described above.

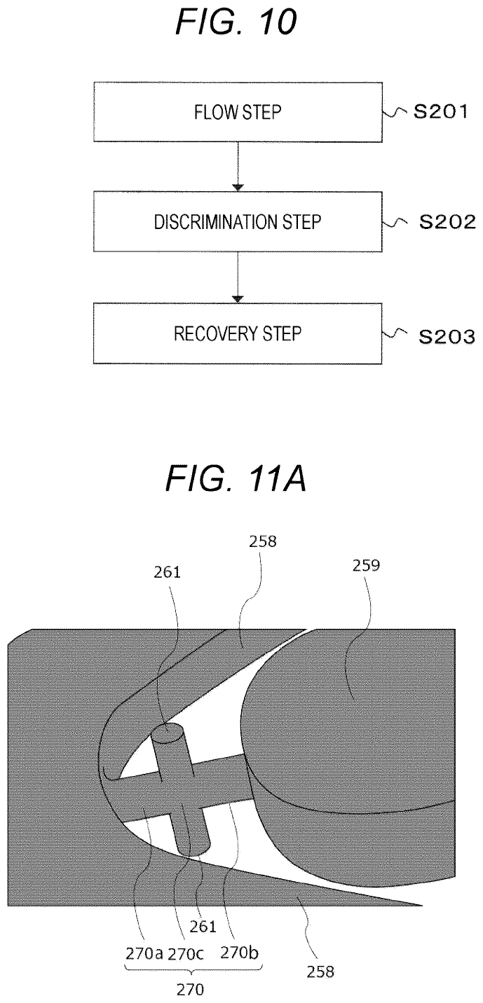

As illustrated in FIG. 10, in order to form an emulsion containing emulsion particles each containing one target bioparticle (the bioparticle P to which the first capturing substance, the secreted substance, and the second capturing substance are bound), for example, in the microchip 250, the following steps may be executed: a flow step S201 of allowing a first liquid containing a bioparticle population including a target bioparticle to flow through the main channel 255; a discrimination step S202 of determining whether or not each bioparticle flowing through the main channel 255 is the recovery target particle; and a recovery step S203 of recovering the recovery target particle into the recovery channel 259. The discrimination step S202 corresponds to the discrimination step described in the item (3-4-1). The recovery step S203 corresponds to the particle isolation step described in the item (3-4-2).

Each step is described below.

### (Flow Step)

In the flow step S201, the first liquid containing the bioparticle population flows through the main channel 255. The first liquid flows in the main channel 255 from a junction 262 toward the particle sorting part 257. The first liquid may be a laminar flow including a sample liquid containing bioparticles and a sheath liquid. In particular, the first liquid may be a laminar flow in which the sample liquid is surrounded by the sheath liquid. The channel structure for forming the laminar flow will be described below.

Note that the sheath liquid may contain, for example, a bioparticle disrupting component such as a cytolytic component. As a result, the component is incorporated into the emulsion particle, and the bioparticle can be disrupted in the emulsion particle in the disruption step described later. The cytolytic component may be a cytolytic enzyme, for example, proteinase K. For example, after cells are captured in emulsion particles containing proteinase K, the cells are lysed by placing the emulsion particles at a predetermined temperature (for example, 37° C. to 56° C.) for 1 hour or less, particularly for less than 1 hour. Note that the proteinase K is active even at 37° C. or lower, but in the case of employing such a lower temperature, incubation may be performed, for example, overnight in consideration of the fact that the cytolytic properties of the proteinase K are deteriorated. In addition, the sheath liquid may also contain a surfactant (for example, SDS, Sarkosyl, Tween 20, Triton X-100, or the like). The surfactant can enhance the activity of the proteinase K.

Furthermore, the sheath liquid does not necessarily contain the bioparticle disrupting component. In this case, the bioparticle may be physically disrupted. As a physical disruption technique, for example, an optical treatment (for example, optical lysis) or a thermal treatment (for example, thermal lysis) may be adopted. The optical treatment may be performed, for example, by forming plasma or cavitation bubbles in the particle by irradiating the emulsion particle with a laser beam. Thermal particle disruption may be performed by heating the emulsion particle.

The microchip 250 is provided with a sample liquid inlet 251 and a sheath liquid inlet 253. From these inlets, a sample liquid containing the bioparticle population and a sheath liquid not containing the bioparticle are introduced into a sample liquid channel 252 and a sheath liquid channel 254, respectively.

The microchip 250 has a channel structure in which the sample channel 252 through which the sample liquid flows and the sheath liquid channel 254 through which the sheath liquid flows are joined at the junction 262 to become the main channel 255. The sample liquid and the sheath liquid join at the junction 262 to form, for example, the laminar flow in which the sample liquid is surrounded by the sheath liquid. A schematic view of the formation of the laminar flow is illustrated in FIG. 7B. As illustrated in FIG. 7B, a laminar flow is formed such that the sample liquid introduced from the sample channel 252 is surrounded by the sheath liquid introduced from the sheath liquid channel 254.

Preferably, in the laminar flow, the bioparticles are arrayed substantially in a line. For example, as illustrated in FIG. 7B, the bioparticles P may be arrayed substantially in a line in the sample liquid. As described above, in the present disclosure, the channel structure forms a laminar flow including bioparticles flowing substantially in a line.

The laminar flow flows through the main channel 255 toward the particle sorting part 257. Preferably, the bioparticles flow in a line in the main channel 255. Therefore, in light irradiation in a detection area 256 to be described below, it becomes easy to distinguish light generated when irradiating one microparticle with light from light generated when irradiating other microparticles with light.

### (Discrimination Step)

In the discrimination step S202, it is determined whether or not the bioparticle flowing through the main channel 255 is the recovery target particle. The discrimination may be performed by the determination unit 295. The determination unit 295 can perform the discrimination on the basis of light generated by the irradiation of the bioparticle with light by the light irradiation unit 291. An example of the discrimination step S202 is described below in further detail.

In the discrimination step S202, the light irradiation unit 291 irradiates the bioparticle flowing through the main channel 255 (in particular, the detection area 256) in the microchip 250 with light (for example, excitation light), and the detection unit 292 detects the light generated by the light irradiation. According to the feature of the light detected by the detection unit 292, the determination unit 295 included in the control unit 293 determines whether or not the bioparticle is the recovery target particle. For example, the discrimination unit 295 may make a determination based on scattered light, a determination based on fluorescence, or a determination based on an image (for example, one or more of a dark field image, a bright field image, and a phase contrast image). In the recovery step S203 described later, the control unit 293 controls the flow in the microchip 250 to recover the recovery target particle into the recovery channel 259.

The light irradiation unit 291 irradiates the bioparticle flowing in the channel in the microchip 250 with light (for example, excitation light). The light irradiation unit 291 may include a light source that emits light, and an objective lens that condenses the excitation light on the microparticle flowing through the detection area. The light source may be appropriately selected by those skilled in the art depending on a purpose of an analysis, and may be, for example, a laser diode, an SHG laser, a solid-state laser, a gas laser, a high brightness LED, or a halogen lamp, or may be a combination of two or more of them. The light irradiation unit may include other optical elements as needed in addition to the light source and the objective lens.

(Discrimination of Sorting Target Based on Fluorescence Signal or/and Scattered Light Signal)

In one embodiment of the present disclosure, the detection unit 292 detects scattered light and/or fluorescence generated from the microparticle by the light irradiation by the light irradiation unit 291. The detection unit 292 may include a condensing lens that condenses the fluorescence and/or scattered light generated from the bioparticle, and a detector. As the detector, PMT, photodiode, CCD, CMOS and the like may be used, but the detector is not limited thereto. The detection unit 292 may include other optical elements as needed in addition to the condensing lens and the detector. The detection unit 292 may further include, for example, a spectroscopic unit. Examples of optical components that form the spectroscopic unit may include a grating, a prism, and an optical filter, for example. The spectroscopic unit can detect, for example, light having a wavelength that should be detected separately from light having another wavelength. The detection unit 292 may convert the detected light into an analog electric signal by photoelectric conversion. The detection unit 292 may further convert the analog electric signal into a digital electric signal by AD conversion.

The signal processing unit 294 included in the control unit 293 may process the waveform of the digital electric signal obtained by the detection unit 292 to generate information (data) regarding the feature of the light used for the determination by the determination unit 295. As the information regarding the feature of the light, the signal processing unit 294 may acquire, from the waveform of the digital electric signal, for example, one, two, or three of the width of the waveform, the height of the waveform, and the area of the waveform. Furthermore, the information regarding the feature of the light may include, for example, time when the light is detected. Processing by the signal processing unit 294 described above may be performed especially in the embodiment in which the scattered light and/or fluorescence is detected.

The determination unit 295 included in the control unit 293 determines whether or not the bioparticle is the recovery target particle, on the basis of the light generated by irradiating the bioparticle flowing in the channel with light.

In the embodiment in which the scattered light and/or fluorescence is detected, the waveform of the digital electric signal acquired by the detection unit 292 is processed by the control unit 293, and then, on the basis of the information regarding the feature of the light generated by the processing, the determination unit 295 determines whether or not the bioparticle is the recovery target particle. For example, in the determination based on the scattered light, the feature of the outer shape and/or the internal structure of the bioparticle may be specified, and it may be determined whether or not the bioparticle is the recovery target particle on the basis of the feature. Moreover, for example, by performing pretreatment on the bioparticle such as a cell in advance, it is possible to determine whether or not the bioparticle is the recovery target particle on the basis of the feature similar to that used in flow cytometry. Furthermore, for example, by labeling the bioparticle such as the cell with an antibody or dye (especially, a fluorescent dye), it is possible to determine whether or not the bioparticle is the recovery target particle on the basis of the feature of a surface antigen of the bioparticle.

(Discrimination of Sorting Target Based on Bright Field Image and/or Phase Contrast Image)

In another embodiment of the present disclosure, the detection unit 292 may acquire a bright field image and/or a phase contrast image generated by the light irradiation by the light irradiation unit 291. In this embodiment, the light irradiation unit 291 includes, for example, a halogen lamp, and the detection unit 292 may include a CCD or a CMOS. For example, the bioparticle is irradiated with light by the halogen lamp, and the CCD or CMOS may acquire a bright field image and/or a phase contrast image of the irradiated bioparticle.

In the embodiment in which the bright field image and/or the phase contrast image are acquired, the determination unit 295 included in the control unit 293 determines whether or not the bioparticle is the recovery target particle on the basis of the acquired bright field image and/or phase contrast image. For example, it may be determined whether or not the bioparticle is the recovery target particle on the basis of one or a combination of two or more of the morphology, size, and color of the bioparticle (especially, the cell).

### (Discrimination of Sorting Target Based on Dark Field Image)

In still another embodiment of the present disclosure, the detection unit 292 may acquire a dark field image generated by the light irradiation by the light irradiation unit 291. In this embodiment, the light irradiation unit 291 includes, for example, a laser light source, and the detection unit 292 may include a CCD or a CMOS. For example, the bioparticle is irradiated with light by a laser, and the CCD or CMOS may acquire a dark field image (for example, a fluorescence image) of the irradiated microparticle.

In the embodiment in which the dark field image is acquired, the determination unit 295 included in the control unit 293 determines whether or not the bioparticle is the recovery target particle on the basis of the acquired dark field image. For example, it may be determined whether or not the bioparticle is the recovery target particle on the basis of one or a combination of two or more of the morphology, size, and color of the bioparticle (especially, the cell).

In any of the “discrimination of the sorting target based on the fluorescence signal or/and the scattered light signal”, the “discrimination of the sorting target based on the bright field image”, and the “discrimination of the sorting target based on the dark field image” described above, the detection unit 292 may be, for example, an imaging element in which a substrate in which a CMOS sensor is incorporated and a substrate in which a digital signal processor (DSP) is incorporated are laminated. By allowing the DSP of the imaging element to operate as a machine learning unit, the imaging element may operate as a so-called AI sensor. The detection unit 292 including the imaging element may determine whether or not the bioparticle is the recovery target particle, for example, on the basis of a learning model. Furthermore, the learning model may be updated in real time while the method according to the present disclosure is performed. For example, the DSP may perform machine learning processing during reset of a pixel array unit in the CMOS sensor, exposure of the pixel array unit, or readout of a pixel signal from each unit pixel of the pixel array unit. As an example of the imaging element that operates as the AI sensor, there may be, for example, the imaging device disclosed in International Publication No. 2018/051809. In a case where the AI sensor is used as the imaging element, the raw data acquired from the image array is learned as it is, so that the speed of the sorting discrimination processing is fast.

The discrimination may be performed, for example, by whether or not the information regarding the feature of the light meets a standard designated in advance. The standard may be a standard indicating that the bioparticle is the recovery target particle. The standard may be appropriately set by those skilled in the art, and may be the standard regarding the feature of the light such as the standard used in the technical field of the flow cytometry and the like, for example.

One position in the detection area 256 may be irradiated with one light, or each of a plurality of positions in the detection area 256 may be irradiated with light. For example, the microchip 250 may be formed so that each of two different positions in the detection area 256 is irradiated with light (that is, there are two positions irradiated with the light in the detection area 256). In this case, for example, it may be determined, on the basis of the light (for example, fluorescence and/or scattered light) generated by irradiating the bioparticle in one position with light, whether or not the bioparticle is the recovery target particle. Moreover, on the basis of a difference between the detection time of light generated by the light irradiation at the one position and the detection time of light generated by light irradiation at another position, the speed of the bioparticle in the channel can also be calculated. For the calculation, a distance between two irradiation positions may be determined in advance, and the speed of the bioparticle may be determined on the basis of a difference between the two detection times and the distance. Moreover, it is possible to accurately predict an arrival time at the particle sorting part 257 described below on the basis of the speed. By accurately predicting the arrival time, it is possible to optimize a timing of forming a flow entering the recovery channel 259. Furthermore, in a case where a difference between an arrival time of a certain bioparticle at the particle sorting part 257 and an arrival time of a bioparticle before or after the certain bioparticle at the particle sorting part 257 is equal to or less than a predetermined threshold value, it can also be determined that the certain bioparticle is not to be recovered. In a case where a distance between the certain bioparticle and a bioparticle before or after is narrow, there is a high possibility that the microparticle before or after is recovered together when the certain bioparticle is suctioned. In a case where there is a high possibility that the bioparticle before or after is recovered together, the recovery of the bioparticle before or after can be prevented by determining that the certain bioparticle is not to be recovered. As a result, the purity of a target bioparticle among the recovered bioparticles can be increased. A specific example of a microchip in which each of two different positions in the detection area 256 is irradiated with light and a device including the microchip is described in, for example, Japanese Patent Application Laid-Open No. 2014-202573.

Note that, the control unit 293 may control the light irradiation by the light irradiation unit 291 and/or the light detection by the detection unit 292. Furthermore, the control unit 293 may control drive of a pump for supplying a fluid into the microchip 250. The control unit 293 may include, for example, a hard disk in which a program for causing the device to execute the isolation step and an OS are stored, a CPU, and a memory. For example, functions of the control unit 293 may be realized in a general-purpose computer. The program may be recorded in a recording medium such as, for example, a microSD memory card, an SD memory card, or a flash memory. A drive (not illustrated) provided in the bioparticle sorting device 200 reads the program recorded in the recording medium. Then, the control unit 293 may cause the bioparticle sorting device 200 to execute the isolation step according to the read program.

### (Recovery Step)

In the recovery step S203, the bioparticle determined to be the recovery target particle in the discrimination step S202 is recovered into the recovery channel 259. In the recovery step S203, the recovery target particle in a state of being contained in the first liquid is recovered in a second liquid immiscible with the first liquid in the recovery channel. As a result, an emulsion containing the second liquid as a dispersion medium and the first liquid as a dispersoid can be formed in the recovery channel 259, and one recovery target particle is contained in each emulsion particle of the emulsion. As a result, a target bioparticle is isolated in a space in the emulsion particle.

For example, as illustrated in FIG. 7B, the recovery target particle P in a state of being contained in the first liquid depicted in white is recovered in the second liquid depicted in gray. As a result, an emulsion particle 290 is formed, and one recovery target particle P is isolated in a space in one emulsion particle 290.

Hereinafter, the recovery step will be described in more detail.

The recovery step S203 is performed in the particle sorting part 257 in the microchip 250. In the particle sorting part 257, the laminar flow that flows through the main channel 255 separately flows to two waste channels 258. The particle sorting part 257 illustrated in FIG. 7A has two waste channels 258, but the number of branch channels is not limited to two. The particle sorting part 257 may be provided with, for example, one or a plurality of (for example, two, three, or four) branch channels. The branch channel may be configured to branch in a Y shape on one plane as in FIG. 7A, or may be configured to branch three-dimensionally.

In the particle sorting part 257, only in a case where the recovery target particle flows, a flow from the main channel 255 into the recovery channel 259 through the connection channel 270 is formed, and the recovery target particle is recovered into the recovery channel 159. FIG. 8 illustrates an enlarged view of the particle sorting part 257. As illustrated in FIG. 8A, the main channel 255 and the recovery channel 259 communicate with each other via the connection channel 270 coaxial with the main channel 255. As illustrated in FIG. 8B, the recovery target particle flows through the connection channel 270 into the recovery channel 259. As illustrated in FIG. 8C, the microparticle that is not the recovery target particle flows into the waste channels 258.

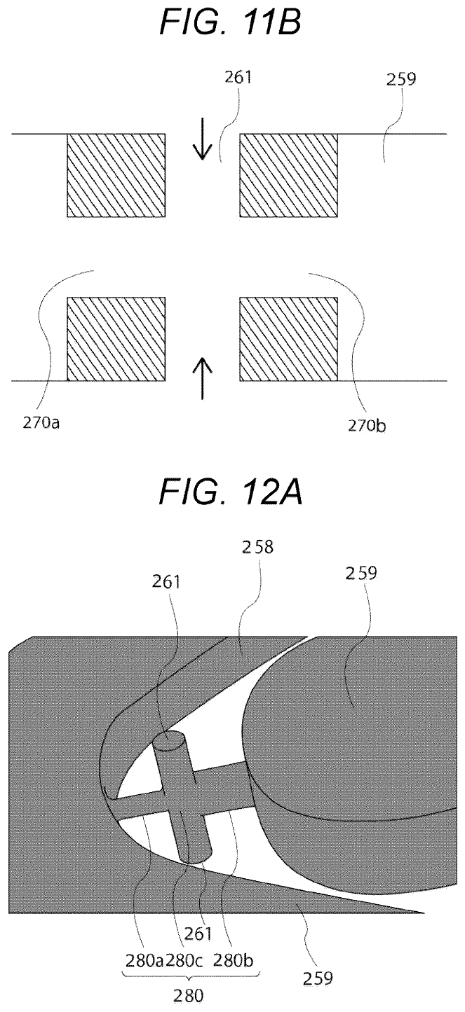

FIGS. 11A and 11B are enlarged views of the vicinity of the connection channel 270. FIG. 11A is a schematic perspective view of the vicinity of the connection channel 270. FIG. 11B is a schematic cross-sectional view on a plane passing through a center line of the liquid supply channel 261 and a center line of the connection channel 270. The connection channel 270 includes a channel 270a on a side of the detection area 256 (hereinafter, also referred to as an upstream side connection channel 270a), a channel 270b on a side of the recovery channel 159 (hereinafter, also referred to as a downstream side connection channel 270b), and a connection 270c between the connection channel 270 and the liquid supply channel 261. The liquid supply channel 261 is provided so as to be substantially perpendicular to the axis of the channel of the connection channel 270. In FIGS. 11A and 11B, two liquid supply channels 261 are provided so as to face each other in substantially the central position of the connection channel 270, but only one liquid supply channel may be provided.

The shape and dimension of the cross-section of the upstream side connection channel 270a may be the same as the shape and dimension of the downstream side connection channel 270b. For example, as illustrated in FIGS. 11A and 11B, both the cross-section of the upstream side connection channel 220a and the cross-section of the downstream side connection channel 220b may be substantially circular with the same dimension. Alternatively, both the two cross-sections may be rectangles (for example, squares or rectangles) having the same dimension.

The second liquid is supplied from the two liquid supply channels 261 to the connection channel 270 as indicated by arrows in FIG. 11B. The second liquid flows from the connection 270c to both the upstream side connection channel 270a and the downstream side connection channel 270b.

In a case where the recovery step is not performed, the second liquid flows as follows.

The second liquid that flows to the upstream side connection channel 270a exits from a connection surface to the main channel 255 of the connection channel 270, and then flows separately to the two waste channels 258. Since the second liquid exits from the connection surface in this manner, it is possible to prevent the first liquid and the microparticle that do not need to be recovered into the recovery channel 259 from entering the recovery channel 259 through the connection channel 270.

The second liquid that flows to the downstream side connection channel 270b flows into the recovery channel 259. As a result, the recovery channel 259 is filled with the second liquid, and the second liquid serves as, for example, a dispersion medium for forming an emulsion.

Also in a case where the recovery step is performed, the second liquid may be supplied from the two liquid supply channels 261 to the connection channel 270. However, due to pressure fluctuation in the recovery channel 259, especially, by generating a negative pressure in the recovery channel 259, a flow from the main channel 255 through the connection channel 270 to the recovery channel 259 is formed. That is, a flow is formed from the main channel 255 through the upstream side connection channel 270a, the connection 270c, and the downstream side connection channel 270b in this order to the recovery channel 259. As a result, the recovery target particle in a state of being encapsulated in the first liquid is recovered in the second liquid in the recovery channel 259. By performing the recovery step, for example, an emulsion may be formed in the recovery channel 259 or in a container connected to a recovery channel end 263, for example, via a channel.

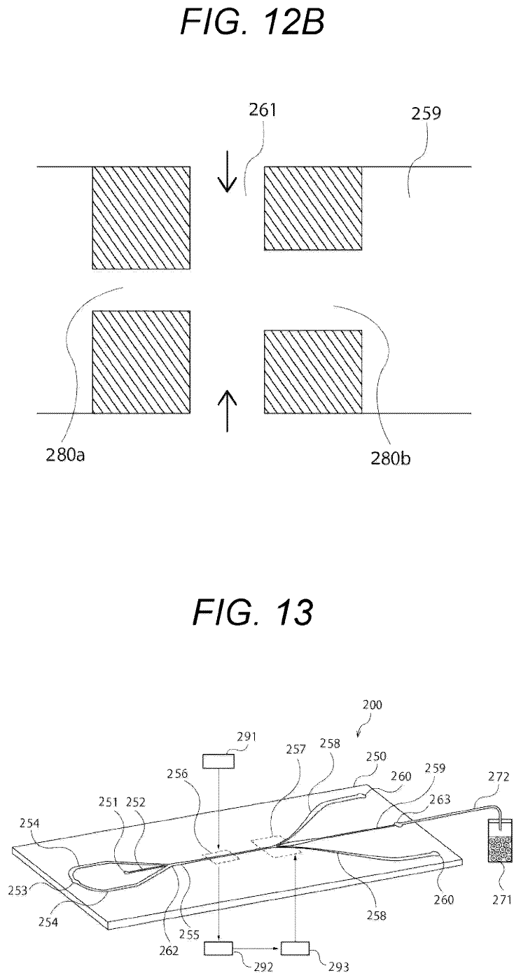

The shape and/or dimension of the cross-section of the upstream side connection channel 220a may be different from the shape and/or dimension of the downstream side connection channel 220b. Examples in which the dimensions of the two channels are different from each other are illustrated in FIGS. 12A and 12B. As illustrated in FIGS. 12A and 12B, a connection channel 280 includes a channel 280a on a side of the detection area 256 (hereinafter, also referred to as an upstream side connection channel 280a), a channel 280b on a side of the recovery channel 259 (hereinafter, also referred to as a downstream side connection channel 280b), and a connection 280c between the connection channel 280 and the liquid supply channel 261. Both the cross-section of the upstream side connection channel 280a and the cross-section of the downstream side connection channel 280b have substantially circular shapes, but the diameter of the cross-section of the latter is larger than the diameter of the cross-section of the former. By making the diameter of the cross-section of the latter larger than that of the former, as compared with a case where the diameters of both are the same, it is possible to more effectively prevent the recovery target particle already sorted into the recovery channel 259 from being emitted to the main channel 255 through the connection channel 280 immediately after the microparticle sorting operation by the negative pressure described above.

For example, in a case where both the cross-section of the upstream side connection channel 280a and the cross-section of the downstream side connection channel 280b are rectangular, by making the area of the cross-section of the latter larger than the area of the cross-section of the former, it is possible to more effectively prevent the already recovered microparticle from being emitted to the main channel 255 through the connection channel 280 as described above.

In the recovery step S203, due to the pressure fluctuation in the recovery channel 259, the recovery target particle is recovered into the recovery channel through the connection channel. The recovery may be performed, for example, by generating the negative pressure in the recovery channel 259 as described above. The negative pressure may be generated, for example, when a wall that defines the recovery channel 259 is deformed by an actuator 297 (especially, a piezo actuator) attached to the outside of the microchip 250. The negative pressure may form the flow entering the recovery channel 259. In order to generate the negative pressure, the actuator 297 may be attached to the outside of the microchip 250, for example, so that the wall of the recovery channel 259 can be deformed. Due to the deformation of the wall, an inner space of the recovery channel 259 is changed, and the negative pressure may be generated. The actuator 297 may be, for example, the piezo actuator. When the recovery target particle is sucked into the recovery channel 259, the sample liquid that forms the laminar flow or the sample liquid and the sheath liquid that form the laminar flow may also flow to the recovery channel 259. In this manner, the recovery target particle is sorted in the particle sorting part 257 and recovered into the recovery channel 259.

The recovery target particle in a state of being encapsulated in the first liquid is recovered in the second liquid immiscible with the first liquid in the recovery channel 259. As a result, as described above, an emulsion containing the second liquid as a dispersion medium and the first liquid as a dispersoid is formed in the recovery channel 259.

The connection channel 270 is provided with the liquid supply channel 261 in order to prevent the bioparticle that is not the recovery target particle from entering the recovery channel 259 through the connection channel 270. The second liquid immiscible with the liquid (the sample liquid and the sheath liquid) flowing through the main channel 255 is introduced from the liquid supply channel 261 into the connection channel 270.

Since a flow from the connection channel 270 toward the main channel 255 is formed by a part of the second liquid introduced into the connection channel 270, it is possible to prevent the bioparticle other than the recovery target particle from entering the recovery channel 259. Due to the flow of the first liquid flowing through the main channel 255 to the waste channels 258, the second liquid formed by the flow from the connection channel 270 toward the main channel 255 flows through the waste channels 258 similarly to the first liquid without flowing through the main channel 255.

Note that, the rest of the second liquid introduced into the connection channel 270 flows to the recovery channel 259. Therefore, the recovery channel 259 may be filled with the second liquid.

The recovery channel 259 may be filled with the second liquid immiscible with the first liquid. In order to fill the recovery channel 259 with the second liquid, the second liquid may be supplied from the liquid supply channel 261 to the connection channel 270. By this supply, the second liquid flows from the connection channel 270 to the recovery channel 259, so that the recovery channel 259 may be filled with the second liquid.

The laminar flow having flowed into the waste channels 258 may be discharged to the outside of the microchip at the waste channel ends 260. Furthermore, the recovery target particle recovered into the recovery channel 259 may be discharged to the outside of the microchip at the recovery channel end 261.

As illustrated in FIG. 13, for example, a container 271 may be connected to the recovery channel end 263 via a channel such as a tube 272. As illustrated in the drawing, in the container 271, an emulsion containing the first liquid containing the recovery target particle as a dispersoid and the second liquid as a dispersion medium is recovered in the container 271. In this way, an emulsion containing emulsion particles into which the bioparticle P having been bound to the first capturing substance, the secreted substance, and the second capturing substance is isolated is obtained. g of FIG. 2D illustrates a state in which the bioparticle P is isolated in an emulsion particle E. The disruption step and the analysis step described later may be executed on the obtained emulsion.

As described above, according to one embodiment of the present disclosure, the bioparticle sorting device 200 may include a channel for recovering the emulsion containing the recovery target particle into the container.

Furthermore, when the recovery channel end 263 is closed and the recovery operation is performed, a plurality of emulsion particles can be retained in the recovery channel 259. After completion of the recovery operation, for example, an assay such as single cell analysis can be continuously performed in the recovery channel 259. For example, the disruption step described later may be performed in the recovery channel 259. Then, the binding between a target capturing molecule and a target substance may be performed along with the disruption step.

As described above, in the microchip used in the present disclosure, the main channel may be branched into the connection channel and the at least one waste channel. The at least one waste channel is a channel through which the bioparticle other than the recovery target particle flows.

Furthermore, as illustrated in FIGS. 7A and 7B, and FIG. 8, in the microchip used in the present disclosure, the main channel, the connection channel, and the recovery channel may be linearly arranged. In a case where these three channels are arranged linearly (especially, coaxially), it is possible to more efficiently perform the recovery step as compared with a case where the connection channel and the recovery channel are arranged at an angle with respect to the main channel, for example. For example, the suction amount required for guiding the recovery target particle to the connection channel can be further reduced.

Furthermore, as illustrated in FIGS. 7A and 7B, in the microchip used in the present disclosure, the bioparticles are arranged substantially in a line in the main channel and flow toward the connection channel. Therefore, the suction amount at the recovery step can be reduced.

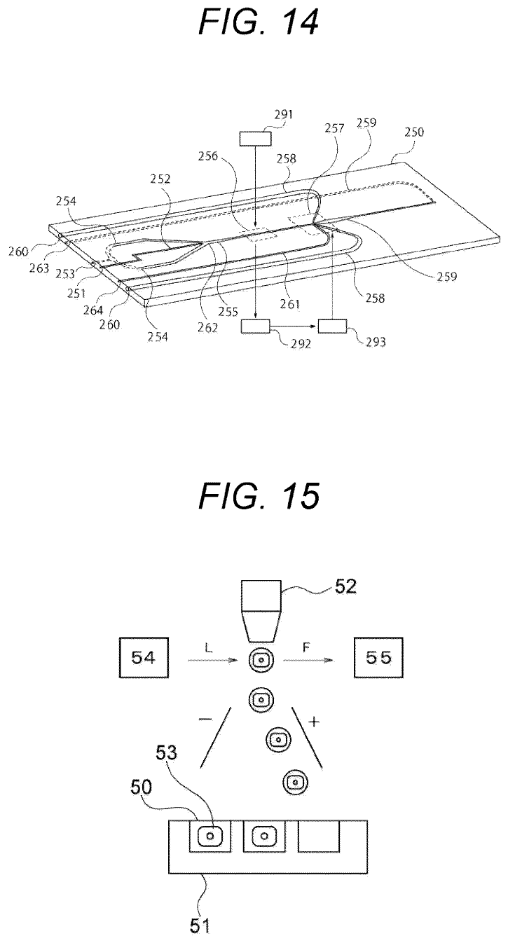

Note that the channel configuration of the microchip used in the present disclosure is not limited to that illustrated in FIG. 7A. For example, in the microchip used in the present disclosure, for example, two or more inlets and/or outlets, preferably all inlets and/or outlets, among inlets into which liquid is introduced and outlets from which liquid is discharged, may be formed on one surface. FIG. 14 illustrates a microchip including the inlet and the outlet which are formed in this manner. In a microchip 350 illustrated in FIG. 14, both the recovery channel end 263 and the two branch channel ends 260 are formed on the surface on which the sample liquid inlet 251 and the sheath liquid inlet 253 are formed. Moreover, an introduction channel inlet 264 for introducing a liquid into an introduction channel 261 is also formed on the surface. In this manner, in the bioparticle sorting microchip 350, all of the inlets into which the liquid is introduced and outlets from which the liquid is discharged are formed on one surface. This facilitates attachment of the chip to the bioparticle sorting device 200. For example, as compared with a case where the inlets and/or outlets are formed on two or more surfaces, connection between channels provided on the bioparticle sorting device 200 and the channels of the bioparticle sorting microchip 350 becomes easy.

Note that, in FIG. 14, a part of the sheath liquid channel 254 is indicated by a dotted line. The part indicated by the dotted line is located in a position lower than that of the sample liquid channel 252 indicated by a solid line (position displaced in an optical axis direction indicated by an arrow), and the channels are not communicated with each other in a position in which the channel indicated by the dotted line intersects with the channel indicated by the solid line. This description also applies to a part indicated by the dotted line in the recovery channel 259 and the branch channel 258 that intersects with the part.

Furthermore, in the present disclosure, the liquid supply channel supplies a liquid (particularly, the second liquid) to the connection channel. Therefore, a flow from a connection position between the liquid supply channel and the connection channel toward the main channel is formed in the connection channel, and it is possible to prevent the liquid that flows through the main channel from entering the connection channel and prevent the microparticle other than the recovery target particle from flowing to the recovery channel through the connection channel. When the recovery step is performed, as described above, for example, due to the negative pressure generated in the recovery channel, the first liquid containing one recovery target particle is recovered into the second liquid in the recovery channel through the connection channel. As a result, an emulsion particle containing one recovery target particle is formed in the second liquid.

Furthermore, in the present disclosure, for example, by driving a piezo actuator at an appropriate timing (for example, at the time at which the bioparticle reaches the particle sorting part 257), a hydrophilic solution containing the bioparticle determined to be the recovery target particle in the determination step is recovered into the recovery channel 259 to form an emulsion particle. In the determination step, it is also possible to determine whether the bioparticle is one microparticle (singlet), a conjugate of two bioparticles (doublet), or a conjugate of three bioparticles (triplet) by determining whether or not the bioparticle is the recovery target particle using, for example, a peak signal and an area signal. It is therefore possible to avoid formation of the emulsion particle in which one emulsion particle contains two or more bioparticles. Therefore, an emulsion particle containing one bioparticle can be formed with high probability and high efficiency. Moreover, since it is possible to avoid formation of an emulsion particle containing a conjugate of two or more bioparticles as described above, it is possible to omit an operation of removing the conjugate product of two or more bioparticles before an operation of forming the emulsion by, for example, a cell sorter or the like.

In the present disclosure, in the isolation step, the emulsion particle may be formed as described above. The bioparticle P to which the first capturing substance, the secreted substance, and the second capturing substance are bound is isolated in the emulsion particle.

### (3-4-2-2) Case of Space in Well

FIG. 15 illustrates a schematic view of an example of a well used for executing the particle isolation step. As illustrated in FIG. 15, for example, a plurality of wells 40 each having a dimension capable of accommodating one bioparticle may be formed in the surface of a substrate 41. A liquid containing the bioparticle population that has undergone the second capturing step described in the item (3-3) is applied to the surface of the substrate 41 from an optional nozzle 42, for example. Thereby, the bioparticle 43 is isolated in a space in the well 40 as illustrated in FIG. 15. In this way, the bioparticle may be isolated in the microspace by placing one bioparticle in a space of one well.

In a case where a liquid containing a plurality of bioparticles is applied to the substrate on which wells are formed as in the example illustrated in FIG. 15, the particle isolation step may be executed without performing the discrimination step described in the item (3-4-2-1).

Furthermore, in a case where the discrimination step described in the item (3-4-2-1) is performed, for example, a device such as a cell sorter or a single cell dispenser that places one bioparticle in one well may be used. Also for the device, a substrate (for example, a plate or the like) on which a plurality of wells is formed may be used for isolating the bioparticle. As the device, a commercially available device may be used. The device may include, for example, a light irradiation unit that irradiates the bioparticle with light, a detection unit that detects the light from the bioparticle, a discrimination unit that determines whether or not the bioparticle is placed into a well on the basis of the detected light, and a distribution unit that distributes the bioparticle determined to be placed into the well, to the well.

The light irradiation unit and the detection unit execute the detection step, and then the discrimination unit executes the discrimination step. The distribution unit includes, for example, a microfluidic chip having a nozzle that forms a droplet containing the bioparticle.

The device places one bioparticle-containing droplet into a predetermined well while operating the position of the microfluidic chip according to the determination result by the discrimination unit. Alternatively, the device controls, according to the determination result by the discrimination unit, the traveling direction of the bioparticle-containing droplet discharged from the nozzle using the charge applied to the droplet. One bioparticle-containing droplet is placed into a predetermined well by the control. In this way, one bioparticle is distributed in one well.

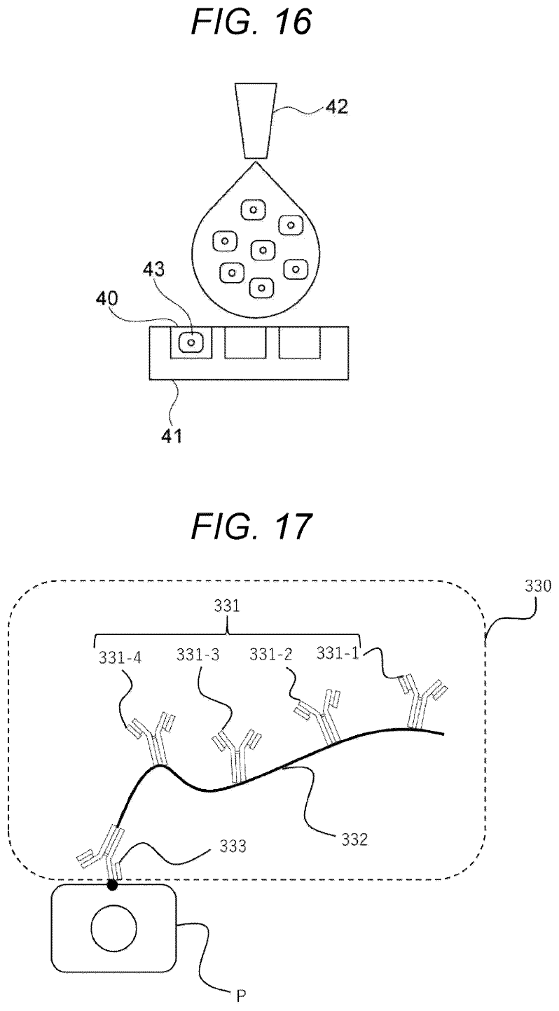

For example, as illustrated in FIG. 16, a bioparticle-containing droplet is discharged from a nozzle 52 provided in the microfluidic chip of the device. The bioparticle contained in the droplet is irradiated with light (for example, laser beam L) by a light irradiation unit 54. Then, the detection step is executed by a detection unit 55, and light (fluorescence F) is detected. The discrimination unit (not illustrated) then executes the determination step on the basis of the detected light. Then, the distribution unit controls, according to the determination result, the traveling direction of the droplet using the charge applied to the droplet. By the control, a droplet containing a target bioparticle is recovered in a predetermined well. As a result, one bioparticle is distributed in one well.

By executing the discrimination step, for example, it is possible to specify the cell population to which the bioparticle belongs, the bioparticle to which the barcode is assigned, or the droplet containing a singlet bioparticle, according to the detection signal. Therefore, only a droplet containing a target bioparticle can be recovered. As a result, it is not necessary to exclude data in the analysis step described later, and the analysis efficiency is improved.

The number of wells provided in one substrate (plate) may be, for example, 1 to 1,000, particularly 10 to 800, and more particularly 30 to 500, but the number of wells may be appropriately selected by those skilled in the art.

As described above, in the present disclosure, the bioparticle P to which the first capturing substance, the secreted substance, and the second capturing substance are bound may be isolated in the well.

### (3-5) Disruption Step

In the disruption step S105, the bioparticle is disrupted in the microspace. The disruption step may be performed under an environment in which a component contained in one bioparticle is not mixed with a component contained in another bioparticle.

Along with the disruption, the conjugate of the first capturing substance, the secreted substance, and the second capturing substance, formed in the second capturing step S103 is dissociated from the bioparticle. Furthermore, along with the disruption, the particle capturing substance 120 is also dissociated from the bioparticle.

Here, the second capturing substance in the conjugate includes a poly A sequence, and the particle capturing substance 120 includes the substance recovery part 122 (for example, a poly T). Therefore, the poly A and the substance recovery part 122 are bound. The second capturing substance includes a capturing substance identifier as described above, and the particle capturing substance includes a particle identifier as described above. Therefore, the capturing substance identifier and the particle identifier are bound to each other via the binding between the poly A and the substance recovery part. Therefore, for example, in the analysis step described later, analysis is possible in a state in which the capturing substance identifier and the particle identifier are associated with each other. More specifically, the secreted substance captured by the second capturing substance can be specified by the capturing substance identifier, and the bioparticle to which the particle capturing substance including the particle identifier has been bound can be specified by the particle identifier. The secreted substance and the bioparticle can be associated with each other accordingly. As a result, information regarding the secreted substance (information regarding the type and/or amount) supplemented by the bioparticle can be associated with the bioparticle, and the secreted substance can be analyzed at a single cell level.

Furthermore, in the disruption step S105, a target substance constituting the bioparticle or a target substance bound to the bioparticle may be captured by the substance recovery part 122 included in the particle capturing substance 120. As a result, a complex of the particle capturing substance 120 and the target substance is formed, and the target substance can be associated with the particle identifier 124 included in the particle capturing substance 120 in the analysis step described later. The complex thus formed is analyzed in the analysis step described later. Therefore, the information regarding the target substance (information regarding the type and/or amount) can be associated with the bioparticle, and the target substance can be analyzed at a single cell level.

The disruption step S105 is preferably executed while the isolation state of the bioparticle in the microspace is maintained. As a result, formation of the conjugate and/or the complex is efficiently performed. Furthermore, it is possible to prevent constituent molecules of the conjugate and/or the complex from binding to molecules outside the microspace.

In a case where the microspace means a space in an emulsion particle, maintaining the isolation state may mean maintaining the emulsion particle, and in particular, may mean that the emulsion particle is not disrupted.

In a case where the microspace means a space in a well, maintaining the isolation state may mean that components in the well (in particular, the bioparticle, the conjugate, the complex, and constituent molecules of the conjugate and/or the complex in the well) remain in the well, and may further mean that components in another well do not enter the well.

The disruption step S105 may be executed by chemically or physically disrupting the bioparticle.

For chemical disruption of the bioparticle, a bioparticle disrupting substance and the bioparticle may be brought into contact with each other in the microspace. The bioparticle disrupting substance may be appropriately selected by those skilled in the art according to the type of the bioparticle. In a case where the bioparticle is a cell, for example, a lipid bilayer membrane disrupting component may be used as the bioparticle disrupting substance, and specifically, a surfactant, an alkali component, an enzyme, or the like may be used. As the surfactant, an anionic surfactant, a nonionic surfactant, an amphoteric surfactant, or a cationic surfactant may be used. Examples of the anionic surfactant include sodium dodecyl sulfate (SDS) and sodium lauroyl sarcosine. Examples of the nonionic surfactant include Triton X-100, Triton X-114, Tween 20, Tween 80, NP-40, Brij-35, Brij-58, octylglucoside, octylthioglucoside, and octylphenoxypolyethoxyethanol. Examples of the amphoteric surfactant include CHAPS and CHAPSO. Examples of the cationic surfactant include cetyltrimethylammonium bromide (CTAB). Also, examples of the alkali component include OH-ions. Also, examples of the enzyme include proteinase K, streptolysin, lysozyme, lysostaphin, zymolase, cellulase, glycanase, and protease. The type of enzyme may be appropriately selected according to, for example, the type of cell (animal cells, plant cells, bacteria, yeast, and the like).

In a case where the microspace is a space in a well, the disruption step may be performed, for example, by placing the bioparticle disrupting substance in each well. Since each well is isolated from each other, the components in the well are maintained in the well even when the disruption is performed.

In a case where the microspace is a space in an emulsion particle, for example, the bioparticle disrupting substance may be introduced into the emulsion particle simultaneously with formation of the emulsion particle. Then, after formation of the emulsion particle, the disruption step of the bioparticle by the bioparticle disrupting substance may be performed.

For physical disruption of the bioparticle, a physical stimulus that disrupts the bioparticle may be applied to the bioparticle. As a treatment for applying the physical stimulus to the bioparticle, for example, an optical treatment, a thermal treatment, an electrical treatment, an acoustic treatment, a freeze-thaw treatment, or a mechanical treatment may be adopted. By these treatments, cells or exosomes can be disrupted. Examples of the optical treatment include plasma formation or cavitation bubble formation by irradiation with a laser beam. Examples of the thermal treatment include a heat treatment. Examples of the acoustic treatment include sonication using ultrasonic waves. Examples of the mechanical treatment include a treatment using a homogenizer or a bead mill. The physical disruption of the bioparticle by these treatments can be applied to both a case where the microspace is a space in a well and a case where the microspace is a space in an emulsion particle. In a case where the microspace is a space in an emulsion particle, among these treatments, an optical treatment, a thermal treatment, an electrical treatment, and a freeze-thaw treatment are particularly suitable. Note that, in order to disrupt the bioparticle while preventing the disruption of the emulsion particle by the acoustic treatment, for example, a surfactant may be added into the emulsion particle, and further, the concentration of the surfactant may be adjusted.

In the disruption step S105, by using the substance recovery part 122 included in the particle capturing substance 120, the secreted substance can be analyzed and the target substance in the cell can be analyzed, and further, the results of these analyses can be associated with the bioparticle. Therefore, the single cell analysis of the secreted substance and the intracellular substance can be simultaneously executed.

The disruption step S105 includes a step of recovering the conjugate and/or the particle capturing substance 120 (particularly, the target substance bound to the particle capturing substance 120) using the substance recovery part 122. The conjugate can be recovered by the substance recovery part 122, and further, the particle capturing substance 120, particularly, the target substance bound to the particle capturing substance 120 can also be recovered.

For example, as illustrated in g of FIG. 2D, the bioparticle P to which the first capturing substance, the secreted substance, and the second capturing substance are bound is isolated in the emulsion particle E. The first capturing substance, the secreted substance, and the second capturing substance are released from the bioparticle P by executing the disruption treatment on the bioparticle P.

Then, for example, as illustrated in h of FIG. 2D, the second capturing substance 170 binds to the particle capturing substance 120. This binding may be based on the binding between the poly A sequence 173 of the second capturing substance 170 and the substance recovery part 122 (poly T sequence in this case) of the particle capturing substance 120. Note that, although the first capturing substance and the secreted substance are not drawn in h of FIG. 2D, after the binding, the secreted substance and the first capturing substance may be continuously bound to the second capturing substance 170, or the secreted substance and the first capturing substance are not necessarily bound to the second capturing substance 170. For example, when the bioparticle P is disrupted, the secreted substance may be released from the second capturing substance 170 or disrupted. Along with this, the first capturing substance may also be released from the second capturing substance 170.

Furthermore, the mRNA inside the bioparticle P is released into the emulsion particle by the disruption of the bioparticle P. Then, the mRNA binds to the substance recovery part (poly T sequence) 122 of the particle capturing substance 120.

As described above, in the disruption step, a conjugate of the particle capturing substance 120 and the second capturing substance 170 is formed in the emulsion particle. Also in the disruption step, a complex of the particle capturing substance 120 and a substance contained in the bioparticle (the target substance described above, particularly, mRNA) may also be formed in the emulsion particle. The conjugate and/or the complex conjugated product is an analysis target in the analysis step described later.

### (3-6) Analysis Step

In the analysis step S106, each bioparticle is analyzed. The analysis may be executed, for example, on the conjugate and/or the complex released by the disruption of the bioparticle in the disruption step S105. For example, as illustrated in i of FIG. 2D, analysis may be executed on the conjugate of the particle capturing substance 120 and the second capturing substance 170. In addition, analysis may be executed on the complex of the particle capturing substance 120 and the substance (the target substance described above, particularly, mRNA) contained in the bioparticle.

The conjugate and the complex each include the recovered substance amplification part and the second recovered substance amplification part. Thus, the analysis in the analysis step S106 may include a nucleic acid amplification step of amplifying the nucleic acid contained in the conjugate and/or the complex, using the recovered substance amplification part and/or the second recovered substance amplification part. In the nucleic acid amplification step, the capturing substance identifier (particularly, nucleic acid, more particularly, mRNA) contained in the conjugate is amplified, and/or the target substance (particularly, nucleic acid, more particularly, mRNA) contained in the complex is amplified. Then, the nucleic acid sequence information is obtained by executing sequencing processing on the amplified nucleic acid.

Here, the capturing substance identifier contained in the conjugate (particularly, sequence information included in the identifier) is associated with the secreted substance. Therefore, the secreted substance can be specified from the nucleic acid sequence information. Furthermore, the target substance contained in the complex is a substance contained in the bioparticle or a substance bound to the bioparticle, and this substance is amplified. Therefore, the target substance can be specified from the nucleic acid sequence information. The secreted substance and the target substance can be specified by the sequencing processing accordingly.

In addition, as described above, the capturing substance identifier contained in the conjugate is bound to the particle identifier via the binding between the poly A and the substance recovery part. The particle capturing substance contained in the complex is also bound to the particle identifier. Therefore, the amplified nucleic acid also includes the sequence of the particle identifier, that is, the nucleic acid sequence information obtained by the sequencing processing also includes information regarding the particle identifier. Therefore, among a plurality of types of nucleic acid sequence information, nucleic acid sequence information including the sequence of the same particle identifier can be specified as being derived from the conjugate (secreted substance) bound to the same bioparticle or the target substance contained in the same bioparticle.

As described above, in the analysis step S106, the information regarding the specified secreted substance and/or target substance may be associated with one bioparticle on the basis of the sequence of the particle identifier.

Since the conjugate and/or the complex include a particle identifier in the disruption step, even in a case where the conjugate and/or the complex derived from different bioparticles respectively present in a plurality of microspaces are collectively analyzed in the analysis step S106, the results of analysis for the conjugate and/or the complex can be associated with the bioparticle from which the conjugate and/or the complex are derived, on the basis of the particle identifier.

For example, in a case where the microspace is a space in a well, the bioparticle disruption products in each well may be separately analyzed, or the bioparticle disruption products of a plurality of wells may be collected as one sample, and the one sample may be collectively analyzed. In the former case, it is easy to associate the bioparticle with the analysis result. Also in the latter case, since the secreted substance or the target substance contained in each bioparticle disruption product is present as a constituent element of the conjugate or complex containing the particle identifier, the analysis result for the conjugate or the complex can be associated with the bioparticle from which the conjugate or the complex is derived.

Furthermore, in a case where the microspace is a space in an emulsion particle, a plurality of emulsion particles may be collectively analyzed, and for example, the entire obtained emulsion may be collectively analyzed. Since the secreted substance or the target substance contained in each bioparticle disruption product is present as a constituent element of the conjugate or complex containing the particle identifier, the analysis result for the conjugate or the complex can be associated with the bioparticle from which the conjugate or the complex is derived. Therefore, the analysis efficiency can be improved.

The analysis step S106 may be performed using an analyzer 1000 as illustrated in i of FIG. 2D. The analyzer 1000 may be, for example, a device that performs sequencing processing on the conjugate and/or the complex. By the sequencing processing, sequence information of nucleic acid, particularly DNA or RNA, more particularly mRNA is obtained. The sequencing processing may be performed by a sequencer, or may be performed by a next-generation sequencer or a sequencer employing the Sanger method. In order to comprehensively perform analysis of a plurality of bioparticles (particularly, a cell population) at a higher speed, the sequencing processing may be performed by a next-generation sequencer.

In order to perform sequencing processing in the analysis step S106, the analysis step may further include a preparation step of preparing a nucleic acid (for example, DNA) to be subjected to sequencing processing, and a purification step of purifying the nucleic acid. By the preparation step and purification step, for example, a library for performing next-generation sequencing processing may be prepared.

The preparation step may include, for example, a cDNA synthesis step of synthesizing cDNA from mRNA. In addition, the preparation step may also include an amplification step of amplifying the synthesized cDNA. After the preparation step, a purification step of purifying the nucleic acid obtained in the preparation step may be performed. The purification step may include, for example, a decomposition treatment of components other than nucleic acid, using an enzyme such as proteinase K. Also in the purification step, a nucleic acid recovery treatment may be performed. In the nucleic acid recovery treatment, for example, a commercially available nucleic acid purification reagent may be used, and examples thereof include magnetic beads such as AMPure XP. Note that, in the purification step, intracellular dsDNA may also be recovered, but the dsDNA can be prevented from being sequenced in the sequencing processing. For example, by incorporating an adaptor sequence for sequencing processing (particularly for next-generation sequencing processing) in the sequence to be amplified (for example, in the second capturing substance and the particle capturing substance), only a nucleic acid including the adaptor sequence can be sequenced.

In the analysis step S106, the secreted substance and/or the target substance may be analyzed for each bioparticle on the basis of the result of the sequencing processing.

For example, in the analysis step S106, the type of the second capturing substance (particularly, the sequence of the capturing substance identifier) and/or the number of the second capturing substances may be determined. The determination may be made on the basis of the sequence of the capturing substance identifier in the sequence determined by the sequencing processing. Thereby, the type and/or number of secreted substances captured by the second capturing substance is determined.

Furthermore, in the analysis step S106, the sequence of the target substance (such as mRNA contained in the cell) and/or the number of copies of each target substance may be determined.

Such analysis of the secreted substance and/or the target substance for each bioparticle may be performed on the basis of the particle identifier in the sequence determined by the sequence processing. For example, a base sequence including a sequence of the same particle identifier is selected from a large number of base sequences determined by the sequence processing. The base sequence including a sequence of the same particle identifier is based on the second capturing substance that has captured the secreted substance bound to one cell and/or the particle capturing substance bound to the component contained in the cell. Therefore, by collecting the analysis results of the secreted substance and/or the target substance for each particle identifier, these substances can be analyzed for each bioparticle.

### 2. Second Embodiment (Reagent Kit for Bioparticle Analysis)

The present disclosure provides a reagent kit for bioparticle analysis, the reagent kit including: a first secreted substance capturing substance including: a first bioparticle binding part configured to bind to a bioparticle; and a first secreted substance binding part configured to bind to a secreted substance generated by placing a bioparticle population including the bioparticle under a predetermined condition; and a second secreted substance capturing substance including: a second secreted substance binding part configured to bind to the secreted substance; and a capturing substance identifier configured to identify a second capturing substance.

The first secreted substance capturing substance is the first capturing substance 130 described in the above section 1. The description regarding the first capturing substance 130 also applies to the first secreted substance capturing substance in the present embodiment. The first bioparticle binding part and the first secreted substance capturing substance are respectively the bioparticle binding part 133 and the secreted substance binding part 131 described in the above section 1. The description regarding the bioparticle binding part 133 and the secreted substance binding part 131 also applies to the first bioparticle binding part and the first secreted substance capturing substance in the present embodiment. The first secreted substance capturing substance may further include a crosslinking part that crosslinks the first bioparticle binding part and the first secreted substance binding part.

For example, the first bioparticle binding part may contain an antigen binding substance that binds to an antigen on the surface of the bioparticle, or a molecule binding substance that binds to a molecule forming a surface membrane of the bioparticle. The antigen binding substance may contain a substance selected from the group consisting of an antibody, an antibody fragment, an aptamer, and a molecularly imprinted polymer. The molecule binding substance may include an oleyl group or a cholesteryl group.

The second secreted substance capturing substance is the second capturing substance 170 described in the above section 1. The description regarding the second capturing substance 170 also applies to the second secreted substance capturing substance in the present embodiment. The second secreted substance binding part and the capturing substance identifier are the second secreted substance binding part 171 and the capturing substance identifier 173 described in the above section 1. The description regarding the second secreted substance binding part 171 and the capturing substance identifier 173 also applies to the second secreted substance binding part and the capturing substance identifier in the present embodiment.

The reagent kit may further include a base material having a surface on which a particle capturing substance including: a second bioparticle binding part configured to bind to the bioparticle; and a particle identifier configured to identify the bioparticle is immobilized. The surface and the base material are the surface 110 and the base material 100 described in the above section 1. The particle capturing substance is the particle capturing substance 120 described in the above section 1. Therefore, the description regarding the surface 110, the base material 100, and the particle capturing substance 120 also applies to the surface, the base material, and the particle capturing substance in the present embodiment.

The reagent kit for bioparticle analysis according to the present disclosure may be used in the bioparticle analysis method according to the present disclosure. As described in the above section 1., among the materials contained in the reagent kit, the combination of the first secreted substance capturing substance and the second secreted substance capturing substance is used for capturing a secreted substance. In particular, the combination is used for capturing the secreted substance in a state of being bound to the bioparticle.

The first secreted substance capturing substance may further include a crosslinking part that crosslinks the bioparticle binding part and the secreted substance binding part. The crosslinking part is the crosslinking part 132 described in the above section 1. The description regarding the crosslinking part 132 also applies to the crosslinking part in the present embodiment.

The reagent kit may further include a base material having a surface on which a particle capturing substance including: a second bioparticle binding part configured to bind to the bioparticle; and a particle identifier configured to identify the bioparticle is immobilized. The surface and the base material are the surface 110 and the base material 100 described in the above section 1. The particle capturing substance is the particle capturing substance 120 described in the above section 1. Therefore, the description regarding the surface 110, the base material 100, and the particle capturing substance 120 also applies to the surface, the base material, and the particle capturing substance in the present embodiment.

### 3. Third Embodiment (Bioparticle Analysis System)

The present disclosure also provides a bioparticle analysis system. The system may include: a first container in which secretion of a secreted substance is induced by placing a bioparticle population including a bioparticle to which a first capturing substance configured to capture a secreted substance is bound under a predetermined condition; a second container in which a secreted substance bound to the first capturing substance and a second capturing substance configured to capture a secreted substance are bound; and a bioparticle processing device that isolates a bioparticle to which the first capturing substance, the secreted substance, and the second capturing substance are bound, into a single particle.

The first container corresponds to the container 140 in which the first capturing step S102 described in the above section 1. is executed. The second container corresponds to the container 150 in which the second capturing step S103 described in the above section 1. is executed.

The bioparticle processing device may be configured to execute the isolation step S104 described in the above section 1. The bioparticle processing device may be, for example, the bioparticle sorting device 200 described in the above section 1.

The bioparticle analysis system of the present disclosure may further include a device configured to execute the cleavage step S114 (particularly, the detection step and/or the linker cleavage step) described in the above section 1. The device may be the stimulus application device described in the above section 1.

In addition, according to one embodiment, the bioparticle analysis system of the present disclosure may include a bioparticle processing device that isolates a bioparticle to which the first capturing substance, the secreted substance, and the second capturing substance are bound, into a single particle. The bioparticle analysis system may also include the reagent kit for bioparticle analysis according to the present disclosure (or any one or more of the materials contained in the reagent kit), in addition to the bioparticle processing device.

The bioparticle analysis system of the present disclosure may include an analyzer that executes the analysis step described in the above section 1. The analyzer may be, for example, a sequencer.

Note that the technology of the present disclosure may have the following configurations.

[1]

A bioparticle analysis method including:
a preparation step of preparing a bioparticle population including a bioparticle to which a first capturing substance configured to capture a secreted substance is bound;
a first capturing step of binding a secreted substance generated by placing the bioparticle population under a predetermined condition, to the first capturing substance; and
a second capturing step of binding the secreted substance bound to the first capturing substance, to a second capturing substance configured to capture the secreted substance.

[2]

The bioparticle analysis method according to [1], in which
the first capturing step includes a treatment step of placing the bioparticle population under a predetermined condition, and
the treatment step is performed while a population state of the bioparticle population is maintained.

[3]

The bioparticle analysis method according to [1] or [2], in which the first capturing step and the second capturing step are performed while a state in which the first capturing substance is bound to the bioparticle is maintained.

[4]

The bioparticle analysis method according to any one of [1] to [3], in which a particle identifier configured to identify the bioparticle is bound to the bioparticle included in the bioparticle population prepared in the preparation step.

[5]

The bioparticle analysis method according to any one of [1] to [4], in which a capturing substance identifier configured to identify the second capturing substance is bound to the second capturing substance.

[6]

The bioparticle analysis method according to any one of [1] to [4], in which the first capturing substance includes a secreted substance binding part and a bioparticle binding part.

[7]

The bioparticle analysis method according to [6], in which the secreted substance binding part is configured to bind to one or two or more of the secreted substances.

[8]

The bioparticle analysis method according to [6] or [7], in which the bioparticle binding part contains an antigen binding substance that binds to an antigen on a surface of the bioparticle, or a molecule binding substance that binds to a molecule forming a surface membrane of the bioparticle.

[9]

The bioparticle analysis method according to any one of [6] to [8], in which the secreted substance binding part is bound to the bioparticle binding part via a crosslinking part.

[10]

The bioparticle analysis method according to any one of [1] to [9], in which the first capturing substance contains an antibody that binds to surfaces of two or more cells of the same or different types.

[11]

The bioparticle analysis method according to any one of [1] to [10], further including an isolation step of isolating the bioparticle included in the bioparticle population into a single particle after the second capturing step.

[12]

The bioparticle analysis method according to [11], further including a disruption step of disrupting the bioparticle after the isolation step.

[13]

The bioparticle analysis method according to [12], in which the disruption step is performed under an environment in which a component contained in one bioparticle is not mixed with a component contained in another bioparticle.

[14]

The bioparticle analysis method according to [13], further including an analysis step of analyzing each of the bioparticles after the disruption step.

[15]

A reagent kit for bioparticle analysis, the reagent kit including:
a first secreted substance capturing substance including: a first bioparticle binding part configured to bind to a bioparticle; and a first secreted substance binding part configured to bind to a secreted substance generated by placing a bioparticle population including the bioparticle under a predetermined condition; and
a second secreted substance capturing substance including: a second secreted substance binding part configured to bind to the secreted substance; and a capturing substance identifier configured to identify a second capturing substance.

[16]

The reagent kit for bioparticle analysis according to [15], in which the first secreted substance capturing substance further includes a crosslinking part that crosslinks the bioparticle binding part and the secreted substance binding part.

[17]

The reagent kit for bioparticle analysis according to [15] or [16], in which the first bioparticle binding part contains an antigen binding substance that binds to an antigen on a surface of the bioparticle, or a molecule binding substance that binds to a molecule forming a surface membrane of the bioparticle.

[18]

The reagent kit for bioparticle analysis according to [17], in which the antigen binding substance contains a substance selected from the group consisting of an antibody, an antibody fragment, an aptamer, and a molecularly imprinted polymer.

[19]

The reagent kit for bioparticle analysis according to [17], in which the molecule binding substance includes an oleyl group or a cholesteryl group.

[20]

The reagent kit for bioparticle analysis according to any one of [15] to [19], further including a base material having a surface on which a particle capturing substance including: a second bioparticle binding part configured to bind to the bioparticle; and a particle identifier configured to identify the bioparticle is immobilized.

### REFERENCE SIGNS LIST

100 Substrate
110 Surface
120 Particle capturing substance
130 First capturing substance
160 Secreted substance
170 Second capturing substance

1. A bioparticle analysis method comprising:
a preparation step of preparing a bioparticle population including a bioparticle to which a first capturing substance configured to capture a secreted substance is bound;
a first capturing step of binding a secreted substance generated by placing the bioparticle population under a predetermined condition, to the first capturing substance; and
a second capturing step of binding the secreted substance bound to the first capturing substance, to a second capturing substance configured to capture the secreted substance.

2. The bioparticle analysis method according to claim 1, wherein
the first capturing step includes a treatment step of placing the bioparticle population under a predetermined condition, and
the treatment step is performed while a population state of the bioparticle population is maintained.

3. The bioparticle analysis method according to claim 1, wherein the first capturing step and the second capturing step are performed while a state in which the first capturing substance is bound to the bioparticle is maintained.

4. The bioparticle analysis method according to claim 1, wherein a particle identifier configured to identify the bioparticle is bound to the bioparticle included in the bioparticle population prepared in the preparation step.

5. The bioparticle analysis method according to claim 1, wherein a capturing substance identifier configured to identify the second capturing substance is bound to the second capturing substance.

6. The bioparticle analysis method according to claim 1, wherein the first capturing substance includes a secreted substance binding part and a bioparticle binding part.

7. The bioparticle analysis method according to claim 6, wherein the secreted substance binding part is configured to bind to one or two or more of the secreted substances.

8. The bioparticle analysis method according to claim 6, wherein the bioparticle binding part contains an antigen binding substance that binds to an antigen on a surface of the bioparticle, or a molecule binding substance that binds to a molecule forming a surface membrane of the bioparticle.

9. The bioparticle analysis method according to claim 6, wherein the secreted substance binding part is bound to the bioparticle binding part via a crosslinking part.

10. The bioparticle analysis method according to claim 1, wherein the first capturing substance contains an antibody that binds to surfaces of two or more cells of the same or different types.

11. The bioparticle analysis method according to claim 1, further comprising an isolation step of isolating the bioparticle included in the bioparticle population into a single particle after the second capturing step.

12. The bioparticle analysis method according to claim 11, further comprising a disruption step of disrupting the bioparticle after the isolation step.

13. The bioparticle analysis method according to claim 12, wherein the disruption step is performed under an environment in which a component contained in one bioparticle is not mixed with a component contained in another bioparticle.

14. The bioparticle analysis method according to claim 13, further comprising an analysis step of analyzing each of the bioparticles after the disruption step.

15. A reagent kit for bioparticle analysis, the reagent kit comprising:
a first secreted substance capturing substance including: a first bioparticle binding part configured to bind to a bioparticle; and a first secreted substance binding part configured to bind to a secreted substance generated by placing a bioparticle population including the bioparticle under a predetermined condition; and
a second secreted substance capturing substance including: a second secreted substance binding part configured to bind to the secreted substance; and a capturing substance identifier configured to identify a second capturing substance.

16. The reagent kit for bioparticle analysis according to claim 15, wherein the first secreted substance capturing substance further includes a crosslinking part that crosslinks the first bioparticle binding part and the first secreted substance binding part.

17. The reagent kit for bioparticle analysis according to claim 15, wherein the first bioparticle binding part contains an antigen binding substance that binds to an antigen on a surface of the bioparticle, or a molecule binding substance that binds to a molecule forming a surface membrane of the bioparticle.

18. The reagent kit for bioparticle analysis according to claim 17, wherein the antigen binding substance contains a substance selected from the group consisting of an antibody, an antibody fragment, an aptamer, and a molecularly imprinted polymer.

19. The reagent kit for bioparticle analysis according to claim 17, wherein the molecule binding substance includes an oleyl group or a cholesteryl group.

20. The reagent kit for bioparticle analysis according to claim 15, further comprising a base material having a surface on which a particle capturing substance including: a second bioparticle binding part configured to bind to the bioparticle; and a particle identifier configured to identify the bioparticle is immobilized.
